(19) United States
(12) Patent Application Publication
(10) Pub. No.: US 2013/0131104 A1
(43) Pub. Date: May 23, 2013

(54) HISTIDINE RICH PROTEIN-2 DIAGNOSTIC TEST FOR CEREBRAL MALARIA

(71) Applicants: Karl B. Seydel, East Lansing, MI (US); Terrie E. Taylor, Traverse City, MI (US)

(72) Inventors: Karl B. Seydel, East Lansing, MI (US); Terrie E. Taylor, Traverse City, MI (US)

(73) Assignee: Board of Trustees of Michigan State University, East Lansing, MI (US)

(21) Appl. No.: 13/636,435

(22) PCT Filed: Mar. 22, 2011
(86) PCT No.: PCT/US11/29431

§ 371 Date: Nov. 30, 2012

Related U.S. Application Data
(60) Provisional application No. 61/316,643, filed on Mar. 23, 2010.

(51) Int. Cl. G01N 33/68

(52) U.S. Cl. CPC G01N 33/6893
USPC 514/305; 435/7.92

(57) ABSTRACT

The present inventions relate to accurately identifying a subset of patients within a larger group with malarial parasitemia. In particular, the present inventions provide compositions and methods comprising a malarial protein, histidine rich pro-tein-2 (HRP-2) for determining the general severity of a malarial infection in patients. Specifically, the inventions provide a rapid test comprising a read-out for HRP-2 levels in bodily fluids for determining whether a comatose patient's disease is a result of malaria as opposed to coma of another cause with incidental parasitemia. Specifically, in one preferred embodiment, a rapid test is contemplated as a quantitative rapid test dipstick. Further, these inventions relate to predictive tests for patients at risk for progression of relatively mild malaria disease to the more life-threatening cerebral malaria in addition to determining the etiology of malaria infections in comatose patients.

### FIELD OF THE INVENTION

The present inventions relate to accurately identifying a subset of patients within a larger group with malarial parasitemia. In particular, the present inventions provide compositions and methods utilizing a malarial protein, histidine rich protein-2 (HRP-2) for determining the general severity of a malarial infection in patients. Specifically, the inventions provide a rapid test comprising a read-out for HRP-2 levels in bodily fluids for determining whether a comatose patient's disease is a result of malaria as opposed to coma of another cause with incidental parasitemia. In particular, a rapid test read-out comprises a numerical cut-off or cut-off range (established by a Receiver Operating Characteristic (ROC) curve done on a population) specific for cerebral malaria patients. Specifically, in one preferred embodiment, a rapid test is contemplated as a quantitative rapid test dipstick. Further, these inventions relate to predictive tests for patients at risk for progression of relatively mild malaria disease to the more life-threatening cerebral malaria in addition to determining the etiology of malaria infections in comatose patients.

### BACKGROUND

Malaria is a devastating parasitic disease in humans resulting in at least 350-500 million newly infected people per year, and approximately 1 million deaths per year, primarily in Africa. The disease mainly affects children under the age of 5 years who have not yet established immunological responses capable of preventing the development of severe malarial disease.

Diagnosis of severe malaria disease is problematic since not every infection of a malarial parasite results in disease, let alone severe malarial disease. Infection with a malaria causing parasite induces production of serum proteins capable of being detected with Enzyme-linked Immunosorbent Assay (ELISA) tests and rapid diagnostic tests. Currently, these tests merely provide a ‘yes or no’ answer as to whether the patient is infected with malaria parasites; these tests do not provide any information relating to the progression of malarial disease or potential prognosis. Thus, in addition to patients with severe malaria, any patient infected with a malarial parasite including asymptomatic parasitized people will result in positive diagnostic tests. Furthermore, these tests are not capable of indicating which patients will progress to develop symptoms of severe malaria disease. Even more problematic for disease treatment, current malaria parasite detection tests are not capable of determining whether patients with symptoms similar to malaria are severe malaria disease (i.e. ‘true malaria’) or the result of nonmalarial origin with a coincidental malaria parasitemia ('false malaria'). Diagnosis of these various disease states remains a challenge, as they are all caused by Plasmodium species.

Methods for diagnosing severe malaria disease from mild disease are available. Those patients that present with severe disease receive a different treatment than those that present with mild disease. Therefore the current standard is to treat each patient who presents in coma with a positive malaria test empirically with the same anti-malarial treatment, specifically with treatments administered as intravenous therapy (IV) antimalarial medications. Oftentimes, investigations into alternate causes of coma are not pursued. Mild malaria is treated with orally administered antimalarial medications. However there are no diagnostic tests for determining which patients with mild disease will progress to severe disease and further there is no diagnostic test available for living patients for determining whether the malaria parasite is the cause of coma in a patient diagnosed with severe malaria with a positive test for a malaria parasite until the disease has progressed to cause retinal degeneration.

When someone becomes infected, they may not feel sick immediately. The majority of patients who do present with malaria-like symptoms, which can resemble influenza or cold symptoms, typically recover after prompt appropriate therapy. However many of the infections occur in rural areas with little to no infrastructure and poor access to health care. Moreover, a subset of infected patients with malarial-like symptoms progress rapidly to a life-threatening syndrome, in particular cerebral malaria; involving coma, convulsions, and organ failure. At the early, mild, stage appropriate triage is needed to determine which cases are likely to be treated locally with oral medicines, and which infections are likely to progress to life threatening disease needing a higher level of care. However there is a lack of accurate diagnostic tests for determining this prognosis (i.e. prediction of a higher potential to progress to a life threatening disease).

Further, in malaria endemic areas, there is a very high incidence of asymptomatic parasitemia in children (as high as 80%, see, Trape, et al., AJTMH, 1994). Therefore the presence of this large parasitized population complicates the diagnosis of cerebral malaria. Children who become comatose for many reasons OTHER than cerebral malaria will often have incidental parasites in their bloodstream. Recently, it was discovered that a significant number of children that look as if they have cerebral malaria actually have other causes of coma and just incidentally have malaria parasitemia (see, Taylor, et al., Nature Medicine 10(2): 143 (2004)). These patients are labeled as having ‘false malaria’ having symptoms of another disease process that is mimicking the symptoms of cerebral malaria. Currently, the best method for distinguishing between true and false cerebral malaria is by an autopsy. The most accurate pre-mortem diagnostic method is the use of indirect ophthalmoscopy to identify malaria specific changes in the retina, i.e. retinopathy. This method requires a highly trained diagnostician rarely available to patients in malaria endemic areas.

Thus a more accurate and timely diagnostic test is needed for diagnosing subsets of malaria infections, in particular for 1) classifying those patients that specifically have cerebral malaria, the most deadly form of the disease, and 2) identifying those patients who are likely to progress to more serious disease and are therefore in need of a higher acuity of clinical care.

### SUMMARY OF THE INVENTION

The present inventions relate to accurately identifying a subset of patients within a larger group with malarial parasitemia. In particular, the present inventions provide compositions and methods utilizing a malarial protein, histidine rich protein-2 (HRP-2) for determining the general severity of a malarial infection in patients. Specifically, the inventions provide a rapid test comprising a read-out for HRP-2 levels in bodily fluids for determining whether a comatose patient's disease is a result of malaria as opposed to another cause with incidental parasitemia. In particular, a rapid test read-out comprises a numerical cut-off or cut-off range (established by a Receiver Operating Characteristic (ROC) curve done on a population) specific for cerebral malaria patients. Specifically, in one preferred embodiment, a rapid test is contemplated as a quantitative rapid test dipstick. Further, these inventions relate to predictive tests for patients at risk for progression of relatively mild malaria disease to the more life-threatening cerebral malaria in addition to determining the etiology of malaria infections in comatose patients.

In one embodiment, the present invention contemplates a kit, comprising, a diagnostic platform, wherein said diagnostic platform is capable of showing an amount of histidine rich protein-2 in blood above a cutoff value (or cut-off range) for identifying a patient having cerebral malaria from a patient with malaria parasitemia that does not have cerebral malaria. In one embodiment, said kit further comprises a device for obtaining blood. In one embodiment, said diagnostic platform is further capable of showing an amount of histidine rich protein-2 above a cutoff value (or range) for identifying a patient having malaria likely to progress to cerebral malaria from a patient with malaria parasitemia that is unlikely to progress to cerebral malaria. In one embodiment, said diagnostic platform is further capable of showing an amount of histidine rich protein-2 above a cutoff value for identifying a patient having cerebral malaria from a patient that does not have cerebral malaria. In one embodiment, said cutoff value of the diagnostic platform consists of a first cutoff value and a second cutoff value. In one embodiment, said diagnostic platform is further capable of showing an amount of histidine rich protein-2 above a first cutoff value and below a second cutoff value for identifying a patient having malaria likely to progress to cerebral malaria from a patient with malaria parasitemia that is unlikely to progress to cerebral malaria. In one embodiment, said diagnostic platform comprises a solid support attached to an antibody reactive with a histidine rich protein-2. In one embodiment, said anti-malaria antibody is an IgM. In one embodiment, said kit further comprises a labeled antibody reactive with a histidine rich protein-2. In one embodiment, said labeled antibody is reactive with a histidine rich protein-2 bound to said antibody attached to a solid support. In one embodiment, said label is a visual label selected from the group consisting of horseradish peroxidase, alkaline phosphatase, phycoerythrin, Texas Red, rhodamine, and a metal. In one embodiment, said label is biotin. In one embodiment, said labeled antibody reactive with a histidine rich protein-2 is an IgG. In one embodiment, said kit further comprises a diagnostic chart showing a representation of a comparative quantitated amount above said second cutoff value for diagnosing true cerebral malaria. In one embodiment, said kit further comprises a diagnostic chart showing a representation of a comparative quantitated amount below said second cutoff value for diagnosing a condition selected from the group consisting of false cerebral malaria, uncomplicated malaria, unlikely to be at risk of developing cerebral malaria, at risk of developing cerebral malaria, and uninfected. In one embodiment, said diagnostic platform comprises a dipstick. In one embodiment, said dipstick reads positive when said quantitated amount of histidine rich protein-2 is above said first cutoff value. In one embodiment, said kit further comprises a positive control antibody. In one embodiment, said positive control antibody is an anti-serum protein antibody. In one embodiment, said kit further comprises a diagnostic chart showing a representation of a comparative quantitated amount below the first cutoff value for diagnosing a condition selected from the group consisting of false cerebral malaria, uncomplicated malaria, unlikely to progress to cerebral malaria, and uninfected. In one embodiment, said kit further comprises a diagnostic chart showing a representation of a comparative quantitated amount below the second cutoff value and above the first cutoff value for diagnosing a condition selected from the group consisting of false cerebral malaria, uncomplicated malaria, and likely to progress to cerebral malaria. In one embodiment, said dipstick reads positive for a patient at risk of developing cerebral malaria when said quantitated amount of histidine rich protein-2 is above said first cutoff value and below said second cutoff value. In one embodiment, said dipstick reads negative when said quantitated amount of histidine rich protein-2 is below said first cutoff value. In one embodiment, said kit further comprises instructions.

In one embodiment, the present invention contemplates a kit comprising a dipstick, said dipstick capable of quantitating HRP-2 in blood, and instructions, said instructions providing a cut-off, above which the quantitative level indicates cerebral malaria.

In one embodiment, the present invention provides a method, comprising: a) providing, i) blood from a comatose human (e.g. a child), ii) an antibody reactive with histidine rich protein-2, and iii) a cutoff value; b) reacting said antibody with said blood under conditions such that the amount of histidine rich protein-2 in the blood is quantitated; c) determining whether the quantitated amount of histidine rich protein-2 is below or above said cutoff value. In one embodiment, the human is a patient and the reacting is done in a hospital, clinic, testing facility or field site. In one embodiment, prior to measurement of HRP-2 in the blood, the comatose patient is tested for meningitis, e.g. by lumbar puncture. In one embodiment, said method further comprises the step of diagnosing cerebral malaria in said patient when the quantitated amount is above the cutoff value. In one embodiment, said method further comprises the step of treating cerebral malaria in said patient when the quantitated amount is above the cutoff value (e.g. treating with antimalaria chemotherapy such as with quinine). In one embodiment, the comatose human's malaria is resistant to chloroquine. In one embodiment, said antibody is attached to a solid support. In one embodiment, said solid support is a dipstick. In one embodiment, said method further comprises the step of diagnosing the absence of cerebral malaria in said child when the quantitated amount is below the cutoff value. In one embodiment, said method further comprises treating said comatose patient for a disease other than cerebral malaria when the quantitated amount is below the cutoff value. In one embodiment, said dipstick reads negative when said quantitated amount of histidine rich protein-2 is above said cutoff value. In one embodiment, said dipstick reads positive when said quantitated amount of histidine rich protein-2 is above said cutoff value. In one embodiment, said cutoff value of the diagnostic platform consists of a first cutoff value and a second cutoff value.

In one embodiment, the present invention contemplates a method, comprising: a) providing, i) blood from a comatose human, and ii) an antibody reactive with histidine rich protein-2, b) reacting said antibody with said blood under conditions such that the amount of histidine rich protein-2 in the blood is quantitated; and c) determining whether the quantitated amount of histidine rich protein-2 is below or above said cutoff value. In one embodiment, the present invention contemplates a method, comprising: a) providing, i) first and second blood samples from a first and a second comatose human, and an antibody reactive with histidine rich protein-2, b) reacting said antibody with said blood under conditions such that the amount of histidine rich protein-2 in the blood is quantitated; c) determining whether the quantitated amount of histidine rich protein-2 is below or above said cutoff value, wherein said first sample is below said cutoff value and second sample is above said cutoff value; and d) treating said first and second comatose human, wherein said first human is treated differently than said second human. In one embodiment, said second human is treated for cerebral malaria and said first human is treated for a different condition.

In one embodiment, the present invention provides a method, method, comprising: a) providing, i) a diagnostic platform, wherein said diagnostic platform is capable of showing an amount of histidine rich protein-2 in blood, ii) a first blood sample obtained from a patient diagnosed with cerebral malaria retinopathy, iii) a second blood sample obtained from a patient, wherein said patient was determined to not have cerebral malaria retinopathy, and b) adding said first blood sample to said diagnostic platform under conditions for obtaining a quantitative value for an amount of histidine rich protein-2 for a patient diagnosed with cerebral malaria retinopathy, c) adding said second blood sample to said solid support under conditions for obtaining a quantitative value for an amount of histidine rich protein-2 patient was determined to not have cerebral malaria retinopathy, and d) analyzing said quantitative values for establishing a cut-off value or cut-off range generated by receiver operating characteristic curve for identifying a patient having cerebral malaria retinopathy from a patient that does not have cerebral malaria retinopathy. In one embodiment, prior to measurement of HRP-2 in the blood, the comatose patient is tested for meningitis, e.g. by lumbar puncture. In one embodiment, said diagnostic platform is selected from the group consisting of an enzyme-linked immunosorbent assay and a dipstick assay. In one embodiment, said quantitative value is selected from the group consisting of a fluorescent measurement and a visual color measurement. In one embodiment, said receiver operating characteristic curve derived cutoff value consists of a sensitivity value and a specificity value. In one embodiment, said receiver operating characteristic curve cutoff value has at least a 90% confidence interval. In one embodiment, said cutoff value of the diagnostic platform consists of a first cutoff value and a second cutoff value.

In one embodiment, the present invention provides a method, comprising, a) providing, i) a diagnostic platform comprising a solid support, wherein said diagnostic platform is capable of showing an amount of histidine rich protein-2 above a cutoff value in the blood for identifying a patient having cerebral malaria from a patient with malaria parasitemia that does not have cerebral malaria, and ii) a blood sample, and b) adding said blood sample to said solid support under conditions for showing an amount of histidine rich protein-2 in said blood, and c) determining whether said amount of histidine rich protein-2 is above or below the cutoff value for identifying a patient having cerebral malaria from a patient that does not have cerebral malaria. In one embodiment, said cutoff value has at least a 90% confidence interval. In one embodiment, said concentration in said blood sample is above said cutoff value for identifying a patient in need of treatment for cerebral malaria. In one embodiment, said concentration is below the cutoff value for identifying a patient in need of treatment for a disease that is not cerebral malaria. In one embodiment, said cutoff value of the diagnostic platform consists of a first cutoff value and a second cutoff value.

In one embodiment, the present invention provides a method, method, comprising: a) providing, i) blood from a patient (e.g. a child) with uncomplicated malaria, ii) an antibody reactive with histidine rich protein-2, and iii) a cutoff value for identifying a patient with uncomplicated malaria that will progress to severe malaria disease; b) reacting said antibody with said blood under conditions such that the amount of histidine rich protein-2 in the blood is quantitated; c) determining whether the quantitated amount of histidine rich protein-2 is below or above said cutoff value. In one embodiment, said cutoff value of the diagnostic platform consists of a first cutoff value and a second cutoff value.

In one embodiment, the cut-off range for distinguishing true cerebral malaria in a comatose patient from coma by other causes is between approximately 10,000 and 20,000 ng/ml HRP-2 protein in the blood, with preferred cut-off values of 10,000, 14,000 and 20,000 ng/ml HRP-2 protein. That is to say, in one embodiment, patients with values (ng/ml) of HRP-2 protein in the blood less than approximately 10,000-20,000 (and more preferably with values less than 20,000 or 14,000 or 10,000) should be tested for non-malaria causes, while patients with values (ng/ml) of HRP-2 protein greater than approximately 10,000-20,000 (and more preferably with values greater than 10,000 or 14,000 or 20,000) should be treated as having true cerebral malaria.

In one embodiment, the cut-off range for predicting that a patient will progress to a more serious condition (rather than improve) is between approximately 20,000 and 40,000 ng/ml HRP-2 protein in the blood, with preferred cut-off values of 20,000, 30,000 and 40,000 ng/ml HRP-2 protein. That is to say, in one embodiment, patients with values (ng/ml) of HRP-2 protein in the blood less than approximately 20,000-40,000 (and more preferably with values less than 20,000 or 30,000 or 40,000) should be treated with the expectation that they will improve, while patients with values (ng/ml) of HRP-2 protein greater than approximately 20,000-40,000 (and more preferably with values greater than 20,000 or 30,000 or 40,000) should be treated with the expectation that their condition is likely to deteriorate (e.g. treated more aggressively).

### DEFINITIONS

To facilitate an understanding of the present invention, a number of terms and phrases as used herein are defined below:

The use of the article “a” or “an” is intended to include one or more.

The term “malaria” as used herein, in general refers to a disease of humans caused by the Plasmodium genus of parasites. People who get malaria, i.e. are infected by malaria causing parasites, are typically very sick with high fevers, shaking chills, and flu-like illness that may progress to a comatose state that is sometimes fatal. Malaria disease encompasses several syndromes, including “severe malaria syndromes” such as cerebral malaria, severe malarial anemia, acidosis, etc.

Thus, the term “cerebral malaria” or “CM” as used herein, refers to one of the malarial syndromes (i.e. a subset of malaria). CM symptoms comprise an unrousable coma not attributable to another cause, such as a post-convulsion state, hypoglycemia, meningitis, etc., in a patient with malarial parasitemia, in particular, parasitemia by P. falciparum parasites. In one embodiment, prior to measurement of HRP-2 in the blood, the comatose patient is tested for meningitis, e.g. by lumbar puncture.

The term “clinically defined CM” refers to a diagnosis consisting of a Blantyre coma score of less than or equal to 2 on admission to a clinic or hospital, no improvement in coma score after hypoglycemia was corrected or within the first 2 hours after admission, P. falciparum parasitemia of any density, no evidence of meningitis (no pathogens cultured from cerebrospinal fluid collected on admission), and no other identifiable explanation for coma.

The term “uncomplicated malaria” or “UCM” refers to a level of disease severity of a patient, including children, who are parasitemic due to infection with any species of Plasmodium, typically P. falciparum, however their parasitemia has not led and is unlikely to lead to an alteration of consciousness.

The term “true cerebral malaria” refers to patients whose altered consciousness/coma is caused by malaria parasitism. True cerebral malaria is currently diagnosed post-mortem by the detection of malaria parasites sequestered in cerebral capillaries, adherence of infected red blood cells to vascular endothelium, and often additional intra- and perivascular pathology (see, Taylor, et al., Nature Medicine, 2004, herein incorporated by reference).

As opposed to “false cerebral malaria” in reference to patients with altered consciousness/coma, in other words, “coma of a non-malarial cause” concurrent with non-contributory parasitemia, such as asymptomatic parasitemia, such as Reye syndrome (diagnosed histologically), ruptured arteriovenous malformation, hepatic necrosis, and multiple factors including severe anemia, pneumonia, meningitis (see, Taylor, et al., Nature Medicine, 2004, herein incorporated by reference), subarachnoid hemorrhage, trauma, metabolic disorders, toxin injestion, postictal febrile seizure, etc.

The term “noncerebral malaria” or “non-cerebral malaria” or “NCM” refers to patients infected with malaria parasites and showing symptomatic malaria without signs of severity or evidence of vital organ dysfunction, including lacking symptoms of altered consciousness/coma.

The term “altered consciousness” or “altered level of consciousness” refers to a loss of awareness and arousal, also covering a spectrum of abnormalities that exist between mild confusion and coma, such as drowsiness, stupor, unconsciousness, etc.

The term “awareness” refers to a state of consciousness related to receiving and processing information communicated by the five senses, including an individual's mind and its mental processes, the physical and biochemical condition of an individual's brain, and the like.

The term “arousal” refers to a state of consciousness regulated solely by physiological functioning. Its primitive responsiveness to the world is demonstrated by predictable reflex (involuntary) responses to stimuli. Arousal is maintained by the reticular activating system (RAS). This is not an anatomical area of the brain but rather a network of structures (including the brainstem, the medulla, and the thalamus) and nerve pathways which function together to produce and maintain arousal.

The term “coma” refers to a state of unconsciousness. A person in a coma cannot be awakened, fails to respond normally to pain, light or sound, does not have sleep-wake cycles, and does not take voluntary actions. A person in a state of coma can be described as “comatose.”

The term “uninfected” refers to patients lacking exposure to malaria parasites and with P. falciparum-negative thin blood smears.

The term “mosquito” in reference to transmitting malaria refers to a female mosquito capable of being a host to a malaria parasite, i.e. Anopheles sp., and then passing the parasites into humans, typically during feeding.

The term “parasite” as used herein, in reference to a “malaria parasite” refers to a microorganism capable of infecting humans and causing malaria, in particular, a Plasmodium falciparum microorganism.

The term “parasitemia” refers to the presence of a parasite in the blood, for example, the presence of proteins in serum or other bodily fluids indicating parasite infection.

The term “histidine-rich protein II” or “histidine-rich protein 2” or “HRP-2” or “HRP2” refers to a water-soluble protein released from parasitized erythrocytes, for example, Pf HRP-2 refers to a water-soluble protein released from Plasmodium falciparum infected red blood cells. In one embodiment, the platform (e.g. dipstick) is developed using recombinant HRP-2 protein. In one embodiment, the platform (e.g. dipstick) comprises recombinant HRP-2 protein.

The term “retinopathy” refers to some form of damage to the retina of the eye.

The term “retinopathy” in reference to “malarial retinopathy” refers to a visual diagnostic test result showing damage to the retina of the eye directly related to the presence of malaria parasites. Testing for cerebral malaria retinopathy consists of a funduscopic examination of the back part of the eye's interior, including the retina, (also known as ophthalmoscopy, including direct and indirect ophthalmoscopy) and specialist examination techniques using an ophthalmoscope or a biomicroscope (slit lamp biomicroscope) with a slit-lamp.

The term “syndrome” refers to a collection of medical signs and symptoms known to frequently appear together but without a known cause, in other words, a syndrome is a collection of symptoms that indicate the presence of a disease.

The term “disease” in reference to malaria is a person infected with malaria parasites, which includes having a syndrome of malaria.

The term “receiver operating characteristic curve” or “receiver operator characteristic curve” or “ROC” refers to a plot of the true positive rate against the false positive rate for determining a possible cut-off point of a diagnostic test. When the area under the curve at a specific point is near 1 there is a higher chance of correct classification. Conversely, when the area under the curve is closer to 0, there is a higher chance of misclassification to the opposite group. An ROC consists of graphing (1—specificity) on the x-axis vs. the sensitivity values (y-axis). A high sensitivity results in low number of false negative cases. A high specificity refers to low number of false positive cases.

The term “cut-off point” or “cutpoint” refers to a number obtained from an ROC representing a balance between sensitivity and specificity of a diagnostic test. A cut-off range can encompass a number of cut-off embodiments, where each represents a different balance between sensitivity and specificity. An operator/manufacturer of the methods and kits described herein can choose a numerical cut-off within the range, e.g. a higher cut-off value in order to reduce the chance of improperly treating patients, e.g. treating patients under the improper assumption that the patient has true cerebral malaria.

The term “receiver operating characteristic curve derived cutpoint” or “cut-off value” or “cutoff value” refers to a statistically derived optimal number for using as a diagnostic tool wherein a mean and standard deviation (SD) of test values are calculated from a group of read-out values from a category of patients. When a patient's test value is less than this cutoff value the patient may be considered negative (i.e. without cerebral malaria) and when a value is greater than or equal to cutoff value then the patient is considered positive (i.e. with cerebral malaria).

The term “sensitivity value” refers to a measure of the proportion of actual positives, which are correctly identified (e.g. the percentage of sick people who are identified as having the condition).

The term “specificity value” or “1-specificity” refers to measures the proportion of negatives, which are correctly identified (e.g. the percentage of healthy people who are identified as not having the condition).

The term “AUC” refers to the Area Under the Curve of a ROC Curve. It is used as a figure of merit for a test on a given sample population and gives values ranging from 1 for a perfect test to 0.5 in which the test gives a completely random response in classifying test subjects. Since the range of the AUC is only 0.5 to 1.0, a small change in AUC has greater significance than a similar change in a metric that ranges for 0 to 1 or 0 to 100%. When the % change in the AUC is given, it will be calculated based on the fact that the full range of the metric is 0.5 to 1.0. The JMP™ or Analyse-It™ statistical package reports AUC for each ROC curve generated. AUC measures are a valuable means for comparing the accuracy of the classification algorithm across the complete data range. Those classification algorithms with greater AUC have by definition, a greater capacity to classify unknowns correctly between the two groups of interest (diseased and not-diseased). The classification algorithm may be as simple as the measure of a single molecule or as complex as the measure and integration of multiple molecules.

The term “false negative” refers to a positive value (for example, a patient with cerebral malaria), which fails to be detected.

The term “false positive” refers to a negative value (for example, a patient without cerebral malaria), which is incorrectly read as positive.

The terms “diagnostic assay” and “diagnostic method” refer to the detection of the presence or nature of a medical or pathologic condition of interest. Diagnostic assays differ in their sensitivity and specificity. Subjects who test positive for a medical condition, such as, for example, lung cancer and are actually diseased are considered “true positives.” Within the context of the invention, the sensitivity of a diagnostic assay is defined as the percentage of the true positives in the diseased population. Subjects that do have the medical condition, such as lung cancer, for example, but are not detected by the diagnostic assay are considered “false negatives”. Subjects who are not diseased and who test negative in the diagnostic assay are considered “true negatives”. The term specificity of a diagnostic assay, as used herein, is defined as the percentage of the true negatives in the non-diseased population.

The term, “diagnostic platform,” as used herein, refers to any device (e.g. dipstick) and/or method that may be used to detect and/or identify a biological organism. For example, a diagnostic platform may be used to detect and/or identify a malaria parasite infection in a human.

The term, “showing an amount” refers to the use of a method for detecting an amount of histidine rich protein-2 based upon visualization of a label attached to histidine rich protein-2.

The term “parasitemia” refers to the presence of a parasite in the blood, for example, the presence of proteins in serum indicating parasite infection.

The term “diagnostic chart” refers to a visual representation of a series of values, including color representations of a value, for example, a range of numerical values, a range of color densities, such as range of light blue to dark blue, a range of light blue to dark violet, specific colors, such as orange, red, etc., wherein each numerical value range or color represents a diagnostic value set by a cut-point. Specifically, a diagnostic read-out chart of the present inventions represents numbers or colors for identifying a cerebral malaria patient as opposed to a noncerebral malaria patient produced using ROC curve cutpoint values of the present inventions.

The term “solid support”, as used herein, refers to any composition and/or material that is capable of immobilizing a compound including, but not limited to, immobilizing an antibody (i.e., for example, an antibody that binds to an HRP2) or an antigen (i.e., for example, an HRP2 protein). A solid support may include, but is not limited to, a membrane (e.g. a charged membrane), plastic, beads, strips, microtiter wells, microchannels, etc.

The present invention, in one embodiment, contemplates measuring HRP-2 levels in the blood (e.g. whole blood, serum, or plasma) using a “dipstick” device. It is not intended that the present invention be limited to a particular design, i.e. simple or complicated. The term “dipstick” refers to a specialized sold support comprising a test strip, such as nitrocellulose, glass fibre, polymer (e.g. polyester), cellulose nitrate, nylon and the like. A simple dipstick generally uses a plastic strip with membrane containing immobilized antibody attached at one end for dipping into a solution either containing or suspected of containing the analyte of interest. When incubated, analyte present in the sample binds to antibody immobilized over membrane. The extent to which the analyte becomes bound to that zone can be determined with the aid of labeled reagents. Typically, the user determines the concentration of the analyte by comparing the colour on the membrane to the colour on an external calibrator, such as a series of coloured examples that are printed on a label. The colour of each example is associated with a particular concentration of the analyte. The colour on the example that most closely approximates the colour on the dipstick provides the user with an approximate concentration of the analyte in the test samples.

The immunochromatographic test strip device is also an embodiment contemplated by the present invention and is a more complicated dipstick that constitutes an improvement over the simple dipstick. This class of devices has an absorbent strip immobilized with receptor (antibody) near the center of a typically rectangular chromatography medium, e.g. filter paper, membrane and having an end portion for contacting a test solution. The strip having a length and width is capable of conveying fluids in a fluid flow direction generally parallel to the length of the strip. They generally exhibits improved sensitivity in analyte detection relative to that of simple dipstick devices by virtue of the analyte concentrating effect achieved by the flow of sample containing the analyte past an immobilized analyte binding zone. A sample that is suspected of containing the analyte of interest is placed at or near one end of a membrane strip followed by the labeled reagent. The label reagent is an second antibody different from the first antibody yet it also binds with specificity to the analyte, is prepared separately and bound to a detectable marker substance to prepare a marker-second antibody complex. The maker-second antibody complex can be premixed with sample prior to addition to the strip or it can be added substantially simultaneously with the sample or it can be added after sample addition. The mixture is allowed to be carried to the opposite end of the membrane strip by a liquid phase that traverses the membrane strip by capillary action. While traversing the membrane strip, if the sample contains analyte it binds to the receptor (either mobile or stationary phase) and the marker is also captured by the trap yielding a complex of (marker)-(second antibody)-(analyte)-(first antibody). Because the marker is detectable, the presence of the marker can be detected by the naked eye, i.e. by means of colour contrasting with the chromatographic medium. Therefore, a coloured mark or the like will be left by the marker at the site at which the first antibody was affixed and thereby it is possible to easily confirm the presence (or absence) of the analyte.

The present invention also contemplates in one embodiment flow through dipsticks. In one embodiment, these devices utilize flow of fluid in a direction which is primarily transverse to the plane of the membrane. A solution containing the target analyte is drawn through the entire membrane area by capillary action of the adsorbent material located adjacent to the membrane. Absorbent material such as cellulose acetate, filter paper, porous polyethylene is capable of absorbing liquid sample in substantially greater amount than that applied during one test. The absorbent body provides a means to collect the sample by providing uniform suction to deliver the sample through the reaction membrane down into the adsorbent body. Thus, the adsorbent body also acts as a reservoir to hold the sample, and various reagents.

Several analytical devices based on flow-through principle have been developed and described in patents which employ a membrane immunoadsorbent in combination with an absorbent pad. The absorbent body, which constitutes the fluid-receiving zone in these devices, can either be in non-continuous contact with the membrane containing immobilized antibody (U.S. Pat. No. 4,246,339, hereby incorporated by reference) or in continuous contact with membrane (U.S. Pat. Nos. 4,366,241, 4,446,232, 4,632,901 and 4,727,019, all of which are hereby incorporated by reference).

It is not intended that the present invention be limited by the nature of the HRP-2 reactive antibodies. Such antibodies are described in U.S. Pat. No. 5,665,552, incorporated herein by reference.

The term “malaria diagnostic antibody” refers to any antibody capable of binding to a protein specifically related to a malaria parasite infection, for example, an anti-HRP2 antibody.

The term “labeling” in reference to a diagnostic test refers to attaching a label to bound components of the test, such as binding a label conjugated antibody to bound protein, such as HRP2 protein bound to a membrane with an anti-HRP2 antibody bridge, where such attaching may be direct or through bridging molecules.

The term “antibody” refers to an immunoglobulin molecule or immunologically active portion thereof, namely, an antigen-binding portion. Examples of immunologically active portions of immunoglobulin molecules include F(ab) and F(ab)′.sub.2 fragments which can be generated by treating an antibody with an enzyme, such as pepsin. Examples of antibodies include, but are not limited to, polyclonal antibodies, monoclonal antibodies, chimeric antibodies, mouse antibodies, human antibodies, humanized antibodies, recombinant antibodies, single-chain Fvs (“scFv”), an affinity maturated antibody, single chain antibodies, single domain antibodies, F(ab) fragments, F(ab)′ fragments, disulfide-linked Fvs (“sdFv”), and antiidiotypic (“anti-Id”) antibodies and functionally active epitope-binding fragments of any of the above.

The term “antibody” refers to polyclonal and monoclonal antibodies. Polyclonal antibodies which are formed in the animal as the result of an immunological reaction against a protein of interest or a fragment thereof, can then be readily isolated from the blood using well-known methods and purified by column chromatography, for example. Monoclonal antibodies can also be prepared using known methods (See, Winter and Milstein, Nature, 349, 293-299, 1991, herein incorporated by reference). As used herein, the term “antibody” encompasses recombinantly prepared, and modified antibodies and antigen-binding fragments thereof, such as chimeric antibodies, humanized antibodies, multifunctional antibodies, bispecific or oligo-specific antibodies, single-stranded antibodies and F(ab) or F(ab).sub.2 fragments. The term “reactive” when used in reference to an antibody indicates that the antibody is capable of binding an antigen of interest. For example, a HRP-2-reactive antibody is an antibody which binds to HRP-2 or to a fragment of HRP-2.

The term “antigen” refers to a protein, glycoprotein, lipoprotein, lipid or other substance that is reactive with an antibody specific for a portion of the molecule.

The terms “antigenic determinant” and “epitope” as used herein refer to that portion of an antigen that makes contact with a particular antibody and/or T cell receptor. When a protein or fragment of a protein is used to immunize a host animal, numerous regions of the protein may induce the production of antibodies that bind specifically to a given region or three-dimensional structure on the protein; these regions or structures are referred to as antigenic determinants. An antigenic determinant may compete with the intact antigen (i.e., the “immunogen” used to elicit the immune response) for binding to an antibody.

The term “positive control antibody” refers to an antibody that will bind with substances present in the blood of humans. In one embodiment, the positive antibody binds to a substance present in the blood of humans. In another embodiment, the positive antibody binds to a malaria parasite present in the blood of an infected human. In other words, the positive control antibody of the present inventions is intended to provide a visual read-out when the diagnostic test of the present inventions is capable of providing an accurate test result. The term “label” in reference to an antibody refers to a molecule conjugated to an antibody capable of providing a color or number for use in the compositions and methods of the present inventions. For example, a dye, including a fluorescent molecule, such as rhodamine, phycoerythrin, fluorescein iso-thiocyanate, Alexa dyes, etc., a visual dye, horseradish peroxidase, alkaline phosphatase, and the like, and part of a read-out label, such as biotin, streptavidin, for use with a read-out molecule, such as horseradish peroxidase, and the like. A label may be a metal molecule, such as gold, silver, and the like.

The terms “antigenic determinant” and “epitope” as used herein refer to that portion of an antigen that makes contact with a particular antibody and/or T cell receptor. When a protein or fragment of a protein is used to immunize a host animal, numerous regions of the protein may induce the production of antibodies that bind specifically to a given region or three-dimensional structure on the protein; these regions or structures are referred to as antigenic determinants. An antigenic determinant may compete with the intact antigen (i.e., the “immunogen” used to elicit the immune response) for binding to an antibody.

The term “visual” refers to a material where an average human has the capability to detect said material by eye.

The term “read-out means” refers to an analytical detection device, such as an optical reader for determining optical densities of fluorescent or colorimetric molecules, for example, an ELISA reader.

The term “software means” refers to a composition for providing statistical analysis for use in the present inventions, for example, a SASS statistical software package.

The terms “biological sample” and “test sample” refer to all biological fluids and excretions isolated from any given subject. In the context of the present invention such samples include, but are not limited to, blood, blood serum, blood plasma, nipple aspirate, urine, semen, seminal fluid, seminal plasma, prostatic fluid, excreta, tears, saliva, sweat, biopsy, ascites, cerebrospinal fluid, milk, lymph, bronchial and other lavage samples, or tissue extract samples. Typically, blood, serum, and plasma are preferred test samples for use in the context of the present invention.

The teen “pinprick” or “pin prick” refers to an insignificant wound yielding blood, for example, a slight puncture made by or as if by a pin.

The term “finger prick” or “fingerprick” or “pin prick” in reference to a device, refers to a needle, pin, syringe, and any device capable of obtaining less than 1 ml of blood, ideally less than 50 ul of blood, and even more ideally, less than 20 ul of blood.

As used herein, the term “comprising” when placed before the recitation of steps in a method means that the method encompasses one or more steps that are additional to those expressly recited, and that the additional one or more steps may be performed before, between, and/or after the recited steps. For example, a method comprising steps a, b, and c encompasses a method of steps a, b, x, and c, a method of steps a, b, c, and x, as well as a method of steps x, a, b, and c. Furthermore, the term “comprising” when placed before the recitation of steps in a method does not require (although it may encompass) sequential performance of the listed steps, unless the content clearly dictates otherwise. For example, a method comprising steps a, b, and c encompasses, for example, a method of performing steps in the order of steps a, c, and b, the order of steps c, b, and a, and the order of steps c, a, and b, etc.

### DESCRIPTION OF THE FIGURES

FIG. 1. shows an exemplary dot plot of the raw data that was used to generate the ROC curve in FIG. 2.

FIG. 2. shows an exemplary ROC curve contemplated for use in certain embodiments (whether compositions and methods, including kits) of the present inventions.

FIG. 3. shows an exemplary diagnostic device with exemplary cut-offs, a first cutoff and a second cutoff, for diagnosing the severity of a malaria infection for one embodiment, and for determining whether a patient disease will progress in severity to true cerebral malaria in another embodiment.

FIG. 4. shows ELISA was performed on diluted plasma samples from cases with autopsy confirmation of disease state with recombinant HRP2 protein as standard to generate quantification. Previously the standard was based up on parasite equivalents/μl.

FIG. 5. shows an analysis of ng HRP2/ml of archived plasma samples from patients who met the clinical case definition for cerebral malaria and had ophthalmological examinations (2000-2008).

FIG. 6. shows the distribution of ng HRP2/ml plasma for patients found with and without malaria retinopathy from a prospective study including all patients admitted with clinically defined cerebral malaria during a single season. Plasma samples from patients who met the clinical case definition for cerebral malaria, had ophthalmological examinations, and were admitted during the 2009 season were analyzed for ng HRP2/ml plasma.

FIG. 7. shows the distribution of HRP2/ml plasma concentration of 102 patients whose clinical outcome was monitored (85 patients who improved and the 17 patients who clinically deteriorated). The prognostic correlation of HRP2/ml plasma concentration to clinical outcome was examined.

FIG. 8. illustrates the AUC of two other markers: peripheral parasitemia and lactate. As shown herein, plasma HRP-2 concentration correlates better with retinopathy-positive CM than these other markers.

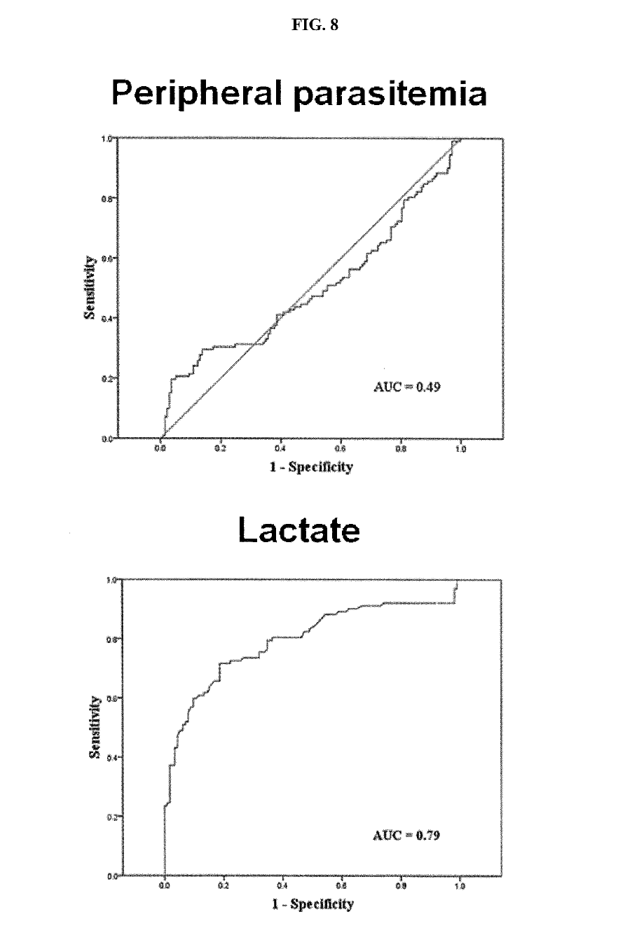

### DETAILED DESCRIPTION OF THE INVENTION

The present inventions relate to accurately identifying a subset of patients within a larger group with malarial parasitemia. In particular, the present inventions provide compositions and methods utilizing a malarial protein, histidine rich protein-2 (HRP-2) for determining the general severity of a malarial infection in patients. Specifically, the inventions provide a rapid test comprising a read-out for HRP-2 levels in bodily fluids for determining whether a comatose patient's disease is a result of malaria as opposed to coma of another cause with incidental parasitemia. In particular, a rapid test read-out comprises a cut-off range or numerical cut-offs (established with a Receiver Operating Characteristic (ROC) curve) specific for cerebral malaria patients. Specifically, in one preferred embodiment, a rapid test is contemplated as a quantitative rapid test dipstick. Further, these inventions relate to predictive tests for patients at risk for progression of relatively mild malaria disease to the more life-threatening cerebral malaria in addition to determining the etiology of malaria infections in comatose patients. In other words, using the cut-off values or ranges described herein, the present invention contemplates embodiments comprising the use of HRP2/ml plasma measurements as a diagnostic method of determining true cerebral malaria and comprising the use of HRP2/ml plasma as a prognostic method for determining those patients that are likely to deteriorate clinically.

Hundreds of millions of people contract malaria each year, primarily in the resource poor countries of sub-Saharan Africa. Malaria is an infectious disease causing the highest fatality rate of parasitic diseases in the world. At least four species of malaria parasites can infect humans: Plasmodium (P.) falciparum, P. vivax, P. ovale, and P. malariae, with P. falciparum infections associated with the more severe forms of malaria disease. Malaria parasitemia occurs when a malarial protozoan parasite is released into the bloodstream of a victim while being bitten by a parasite-carrying mosquito. Because the malaria parasite is found in red blood cells of an infected person, malaria can also be transmitted through blood transfusion, organ transplant, or the shared use of needles or syringes contaminated with blood. Malaria may also be transmitted from a mother to her unborn infant before or during delivery (i.e. “congenital” malaria). After an incubation period of one to four weeks, initial malaria symptoms (i.e. malaria disease) begin. These usually include a fever, headaches, vomiting, chills, and general malaise, similar to the flu. These disease symptoms are caused when the parasite's products are released into the bloodstream.

The form of malaria that affects the brain, cerebral malaria, is caused by Plasmodium falciparum, the mostly deadly of the four malarial (malaria) parasite species. According to Charles Newton, M.D., a physician and researcher with the KEMRI-Wellcome Trust Research Programme in Kilifi, Kenya, more than 2 billion people are exposed to P. falciparum induced malaria in the world annually. Over 500 million episodes of the disease occur each year, some of them repeated illnesses in the same person. These infections result in more than one million deaths, making P. falciparum induced malaria the most fatal parasitic disease in the world. Young children in sub-Saharan Africa bear the brunt of this burden. Infection with the parasite can result in a wide spectrum of disease states. Many individuals can be infected with the parasite but show no symptoms of disease at all. The majority of patients that do show disease symptoms have a mild, or uncomplicated, form of the disease—manifested by fevers and chills, headaches, and vomiting. It is only a small subset of those infected that lead to cerebral malaria, a form of the disease that often manifests itself with convulsions and a loss of consciousness. Children in endemic areas are infected with malaria often, in many areas several times a year. Children that have multiple episodes of the asymptomatic or uncomplicated disease will develop a “clinical immunity” to the severe cerebral form of the disease. This results in the majority of the disease burden lying in the young—those that have not yet developed immunity. Malaria doesn't induce a ‘sterilizing immunity,’ like measles or smallpox, where you get it once and never get it again,” says David Sullivan, M.D., an associate professor at the Johns Hopkins Bloomberg School of Public Health. He explains that as the body develops antibodies that fight malarial infection, the parasites lose the ability to attach to the lining of the blood vessels in numbers as great as in severe P. falciparum disease. Thus, antibodies to malarial parasites and their proteins do not totally prevent a person from contracting malaria again. However, antibodies may keep the number of infected blood cells below the threshold that would cause the more severe forms of the disease. Children under the age of six years and people who are not native to areas where malaria is pervasive have not yet developed this immunity by building up protective antibodies, and so they are left susceptible to the more severe manifestations of the disease, such as cerebral malaria.

People who have little or no immunity to malaria, such as young children or travelers coming from areas with no malaria, are more likely to become very sick and die. People living in rural areas who lack resources and access to health care are at greater risk for this disease. As a result of all these factors, an estimated 90% of deaths due to malaria occur in sub-Saharan Africa; according to the CDC, most of these deaths occur in children under 5 years of age.

Those individuals without well-developed immunity are susceptible to the deadly, cerebral form of the disease. Autopsies of cerebral malaria patients revealed that parasite-infected red blood cells attach in large numbers to the circulatory vessels of the brain. Prompt treatment with intravenous anti-malarial therapy, as well as fluids, and anti-convulsants, is critical in cerebral malaria. Even with the best hospital care, mortality is between 15 and 20 percent.

Children in coma in Africa are traditionally assumed to have cerebral malaria, until proven otherwise. With proper, prompt treatment, the majority of these cerebral malaria patients will recover in three to five days. However, over the next three to seven years, approximately a quarter of those children who did recover will show impairment in memory, attention, and other cognitive skills.

This blanket diagnosis of cerebral malaria is made for several reasons. First, prior to studies by the inventors it was believed that few cases of coma in children had nonmalarial causes. Secondly, proving diagnostically that comatose children have cerebral malaria or not is highly problematic and since treatment must be done immediately for optimal effectiveness these children are immediately treated empirically.

Cerebral malaria is the most common complication and cause of death in severe P. falciparum infection. However the combined factors of merely being infected by P. falciparum and being in a coma was recently discovered to not be an accurate diagnostic determination for cerebral malaria. In fact, the inventors recently determined that at least 23% of deceased children assumed to have cerebral malaria actually died from other causes. This finding was based on a lack of finding parasites sequestered in cerebral capillaries (see, Taylor, et al., Nature Medicine 10(2): 143 (2004), herein incorporated by reference) and higher concentrations of parasite-derived lactate dehydrogenase in tissue samples (see, Seydel, et al., The Journal of Infectious Diseases 194:208 (2006), herein incorporated by reference) in children with retinopathy proven malaria than in those children without retinopathy. Thus the inventors estimated that upwards of a quarter of comatose children are being treated for the wrong medical condition. Thus, determination of the actual cause of coma can be complicated by the fact that large portion of children will have malaria parasites in their blood without having symptoms caused directly from these parasites. Determination of the subset of comatose patients that truly have cerebral malaria (true cerebral malaria), as opposed to coma from an alternate cause (for example, false cerebral malaria) would greatly aid in the diagnosis and treatment of these patients. Thus a false result would lead the clinician to search more diligently for other causes of coma. Therefore, in one embodiment of the present inventions, the inventors provide a method of rapidly identifying cerebral malaria in time for further clinical investigation and alternate treatment before a terminal outcome.

Parasite-derived lactate dehydrogenase levels in tissues provided an indicator of cerebral malaria (Seydel, et al., The Journal of Infectious Diseases 194:208 (2006), herein incorporated by reference). Thus in one embodiment, the invention's methods for detecting cerebral malaria are exemplified by an antibody based assay for detecting a protein associated with infection by a malarial parasite. Both pLDH and the HRP-2 protein used in this application are parasite proteins that are released from the parasite into the bloodstream of the patient. Both can be used diagnostically to determine the presence of Plasmodium parasites in the bloodstream. The observation of unique levels of production of these proteins combined with the varying levels of clearance by the human system result in varying half-lives of these proteins in the bloodstream.

Therefore, the invention is further premised on the surprise discovery, that unlike pLDH, an antibody based assay of a serum protein, pHRP-2, showed a highly statistical correlation with disease state. Further, HRP-2 levels were also discovered by the inventors to have a highly statistical correlation with a subgroup of deceased patients with retinopathy indicative of cerebral malaria as the cause of death. Thus, in one embodiment, the invention's methods for detecting cerebral malaria are exemplified by an antibody-based assay for detecting a serum protein associated with infection by a malarial parasite. In one embodiment, a serum protein is indicative of the parasite load of a patient. In a preferred embodiment, the serum protein is a HRP-2.

Using this surprising information, the inventor's generated ROC curves for determining whether a highly statistically significant cut-off point would be generated for distinguishing a cerebral malaria patient from a patient with malaria parasitemia and nonmalarial forms of comatose conditions (based upon post-mortem evaluation).

A diagnosis of cerebral malaria for this study was based upon clinical presentation prior to death and a retinal exam (ophthalmoscopy) for identifying retinopathy associated with malaria patients. In particular, a large, prospective autopsy study of children dying with cerebral malaria in Malawi found that malarial retinopathy was better than any other clinical or laboratory feature in distinguishing malarial from non-malarial coma. The malarial retinopathy consists of four main components: retinal whitening, vessel changes, retinal hemorrhages, and papilledema. The first two of these abnormalities are specific to malaria, and are not seen in other ocular or systemic conditions. See, Beare et al., Malarial Retinopathy: A Newly Established Diagnostic Sign in Severe Malaria. Am. J. Trop. Med. Hyg. 2006; 75(5):790-797).

Although considered accurate, methods of determining malarial retinopathy require the use of a delicate and relatively expensive medical device, an indirect ophthalmoscope. The use of this diagnostic test in the sub-Saharan location is limited—both by a lack of finances (both from patients and for clinics) in addition to a lack of trained diagnosticians (specialists) to conduct and interpret this diagnostic test. Conversly, moving sick and comatose children from isolated areas to a limited number of clinics with access to diagnostic ophthalmoscope equipment and trained specialists for identifying retinopathy, is extremely problematic due to a lack of finances and a lack of transportation that would transport the child to the clinic within the time frame for treatment based upon a specific diagnosis.

Thus the inventors further contemplate a device for determining the presence of cerebral malaria patients, including comatose children. In another embodiment, the device can provide a determination of a lack of cerebral malaria in malaria patients. In yet another embodiment, the device can provide a determination of cerebral malaria in a subgroup of patients with malaria parasitemia. In yet a further embodiment, the device can provide a determination of whether a patient is infected with a malaria parasite, such that the device further comprises an antimalarial parasite antibody for providing a positive versus negative result. Alternatively, in another embodiment, the device of the present inventions can provide an additional determination of whether a patient is infected with a malaria parasite, such that the device further comprises an antimalarial parasite antibody for providing a positive versus negative result.

The inventors demonstrated several surprising discoveries during the development of the present inventions. In particular, the inventors found after autopsy analysis of child patients with a clinical diagnosis of cerebral malaria prior to death, 23% of the 31 children autopsied had actually died from other causes. The remaining bodies had parasites sequestered in cerebral capillaries. Further, 75% of these deaths of children that had died from cerebral malaria had additional intra- and perivascular pathology. Currently, retinopathy is the one clinical sign used for distinguishing malarial from nonmalarial coma. This new information on causes of death in children with malarial symptom may alter methods for treating malaria patients, changes in designing clinical trials and changing the methods for assessing malaria-specific disease associations.

Even more importantly, higher plasma HRP2 levels strongly correlated with the presence of retinopathy, such that the inventors discovered that HRP2 level determination and analysis was a new diagnostic tool that could replace problematic ophthalmic determination of retinopathy. Further, this discovery is contemplated to allow the development of methods for separating patients with cerebral malaria from patients with malaria infection but without cerebral malaria for guiding treatment decisions. In other words, compositions and methods of the present inventions were developed for identifying a subgroup of patients with malarial parasitism, in particular for identifying patients with cerebral malaria. Thus, in one embodiment, a serum protein is HRP-2. A test of the present inventions will be capable of quantitating the amount of a parasite protein that is present in a patient's blood. The inventors have determined that the levels of this protein can be used to distinguish those children with cerebral malaria from those children with parasites and a nonmalarial disease.

The invention and elements for Use in the present inventions are further described under (A) Proteins, (B) Assays (C) Antibodies binding to proteins related to malarial parasite infections, (D) Generating ROC curves, and (E) Exemplary Contemplated Devices of the present inventions, i.e. Dipsticks And Other Solid Supports.

A. Proteins.

The inventors contemplate measuring the levels of proteins highly correlated with parasite load. In one embodiment, the protein of interest for use in embodiments of the present inventions correlates to a subgroup of patients with malaria parasitemia. In a preferred embodiment, serum levels of the protein of interest for use in embodiments of the present inventions identify patients at risk for disease progression to cerebral malaria. In another preferred embodiment, serum levels of the protein of interest for use in embodiments of the present inventions identify patients with cerebral malaria. In yet a further embodiment, serum levels of the protein of interest correlate with the observation of malaria retinopathy. An exemplary serum protein is histidine-rich protein 2 (HRP2) serum proteins specific induced by Plasmodium falciparum.

Additional proteins and antibodies for binding to these proteins are contemplated in compositions and methods of the present inventions. These additional proteins are contemplated for use as controls for validating tests of the present inventions. As one example, a positive control test

B. Assays.

An assay for use in embodiments of the present inventions includes any assay capable of measuring a specific protein of interest, such as HRP2. In one embodiment, a solid based assay would be used. An exemplary solid based assay is a sandwich ELISA. A sandwich ELISA used in the Experimental section had test wells of a 96 well plate coated with an anti-HRP2 capture antibody, i.e. an IgM. Serum believed to contain HRP2 molecules were incubated in the capture antibody coated wells under conditions for allowing capture of the HEP2 molecules. After rinsing unbound proteins, a second anti-HRP2 antibody was added to each well, i.e. an IgG1, conjugated to a read-out molecule, then incubated under conditions for allowing optimal binding of the read-out antibody. The wells were rinsed again. When the read-out molecule is a fluorescent molecule, an optical reader for generating values is used to read the optical density of the wells. When the read-out molecules need a binding partner for visualization, the wells are incubated in the appropriate material. For example, if the read-out molecule is horseradish peroxidase, then the wells require incubation in a HRP substrate for the read-out, in another example, if the read-out molecule is an alkaline phosphatase molecule, then the wells require incubation in an AP substrate prior to the read-out.

C. Antibodies.

Antibodies for use in compositions and methods of the present inventions may be obtained from several sources. First, antibodies may be purchased commercially, examples of antibodies for use in embodiments of the present inventions, such as anti-HRP2 mouse IgM, kappa (Clone 3565) and anti-HRP2 mouse IgG1 (Clone 3561) directly conjugated to horse radish peroxidase, commercially available from several places, including Thermo Scientific Pierce Antibodies, Rockford, Ill.; GenWay Biotech, Inc. San Diego, Calif.; and ICL, Inc. (Immunology Consultants Laboratory, Inc.) Newberg, Oreg. These exemplary antibodies were successfully used in ELISA assays demonstrating a high correlation between a titration curve of a certain number of parasites and the amount of HRP2 in the culture supernatant (Noedl, et al., ‘Antimicrobial Agents And Chemotherapy,’ August 2005, p. 3575-3577, herein incorporated by reference).

Antibodies may be generation from immunogens (antigens, such as whole proteins, immunogenic fragments, specific epitopes and the like, for generation of polyclonal antibodies and for producing hybridomas).

Exemplary hybridomas may be produced by well-known methods. Immunogens for use in immunizations are prepared using well-known methods. Immunizations may be done with Balb/c mice immunized with the protein of interest, for example, HRP2. The immunogens employed are suspended in Freunds Incomplete Adjuvant at a concentration of 1 ug/ul. A total of 200 ug is injected subcutaneously every 2 weeks until the serum titer of the mouse is half-maximal at a dilution of 2×10⁻⁴ as judged by ELISA with 50 ng of HRP2 attached per well in the solid phase.

Hybridoma Production and Clonal Selection.

Once the desired serum titer is attained, immune spleens are removed from the mice, dissociated, and fused with SP2/o myeloma cells. The resultant cell suspension is plated in 96 well plates, HAT selected and cultured for 10-14 days to allow clonal cell growth. Initial clonal selection is performed by incubating supernatants from each clone in an ELISA well, with HRP2 attached. Clonal supernatants from oligomer-immunized mice that are positive on the oligomer-attached plate but negative (or exhibit a two-fold or greater signal diminution) on the HRP2-attached plate are selected for further subcloning. This dual selection protocol is repeated for screening fusion of splenocytes obtained from HRP2-immunized mice. In this case, clones are selected that bind to HRP2.

Subcloning and Antibody Production.

Parent clones are subcloned 3-4 times to assure monoclonality and allow the hybrids to stabilize. Antibodies are isotyped and the stable clones are adapted to serum-free medium and placed in a bioreactor for antibody expression. Purified, homogenous monoclonal antibodies are then stored at 1 mg/ml in borate buffered saline containing 50% glycerol.

D. Receiver Operating Characteristic Curve (ROC).

Receiver Operating Characteristic curve (or Receiver Operator Characteristic (ROC) curve) refers to a plot of the true positive rate against the false positive rate for different possible cutpoints of a diagnostic test. An ROC curve demonstrates several things:
1. It shows the tradeoff between sensitivity and specificity (any increase in sensitivity will be accompanied by a decrease in specificity).
2. The closer the curve follows the left-hand border and then the top border of the ROC space, the more accurate the test.
3. The closer the curve comes to the 45-degree diagonal of the ROC space, the less accurate the test.
4. The slope of the tangent line at a cutpoint gives the likelihood ratio (LR) for that value of the test.
5. The area under the curve is a measure of test accuracy.

Test values are plotted to generate an ROC curve that is representative of that particular cutoff. The calculated AUC for each test represents the “goodness” of the test or model. Just as in any diagnostic application, the higher the AUC, the better the assay, or in this case the model. A similar procedure is carried out with a second but smaller subset of the data to validate the results. Models that have similar performance in both the training and validation sets are deemed to be optimal and are hence chosen for further analysis and/or validation.

E. Diagnostic Platforms, Dipsticks & Other Solid Supports, and Readouts.

Another diagnostic platform for the identification of malaria infections comprising HRP2 types and subtypes comprises devices and materials enabling the detection of HRP2 in biological materials, and is particularly suited for screening large amounts of samples. One embodiment comprises one or more HRP2-specific monoclonal antibodies and a solid support. In one embodiment, the solid support comprises a microtiter plate, wherein the microtiter plate is optionally coated with the monoclonal antibodies. In another embodiment, the solid support comprises a test strip, wherein the test strip is optionally coated with the monoclonal antibodies. In one embodiment, the test strip comprises nitrocellulose. In one embodiment, the test strip comprises a secondary anti-mouse antibody that is coupled with an enzyme and its substrate or any other molecular compound for a detection reaction (i.e., for example, a peroxidase-labeled anti-mouse IgG antibody, TMB or any other peroxidase substrate).

In another embodiment, the method provides a dipstick format. In some embodiments, the dipstick does not require radioactive tracers, enzymes or substrates. In other embodiments, the dipstick does require radioactive tracers, enzymes, and/or substrates. In one embodiment, the method may require no more than a single step to provide an amount of HRP2 (i.e. a readout) for use with cutoff points of the present inventions. This one-step procedure involves the capture of an HRP2 protein with one of the HRP2 specific MAbs that are immobilized on a solid support (i.e., for example, a nitrocellulose test strip). In another embodiment, the method may require at least one step. Yet other embodiments, the method may require two or more steps to provide an amount of HRP2. These captured HRP2 proteins may be detected directly by a second antibody. In one embodiment, a detector complex results in the formation of colored spots on the test strip that are visible in less than 30 minutes depending on the concentration of the test sample. The spots are a permanent record of the test result and, longer exposures increase the sensitivity of the test without generating higher background, for example, Korth et al., “Immunological detection of prions” U.S. Pat. No. 6,765,088 (2004), herein incorporated by reference.

The biological material containing the HRP2 sample can be insoluble or soluble in buffer or body fluids. The biological material can be derived from any part of the body (i.e., for example, from the brain, or tissue sections). Homogenates may be prepared or any body fluid (i.e., for example, cerebrospinal fluid, urine, saliva, or blood). In the case of body fluids, fluid-resident cells (i.e., for example, white blood cells) can be purified and analyzed either in immunohistochemistry or as a homogenate. Exemplary contemplated devices, device parts, and methods of providing an amount of protein from a sample of the present inventions include but are not limited to descriptions provided in the following publications: United States Patent Application Nos. 20020123671 “Method and apparatus for monitoring biological properties;” 20050196812 “Determining the estimated date of embryo implantation and related dates;” 20090047673 “Miniaturized lateral flow device for rapid and sensitive detection of proteins or nucleic acids;” U.S. Pat. Nos. 7,220,597; 7,141,212; 6,951,631; 6,703,216; 6,585,663; 6,562,631; 6,454,726; 6,451,619; 6,234,974; and the like, all of which are herein incorporated by reference.

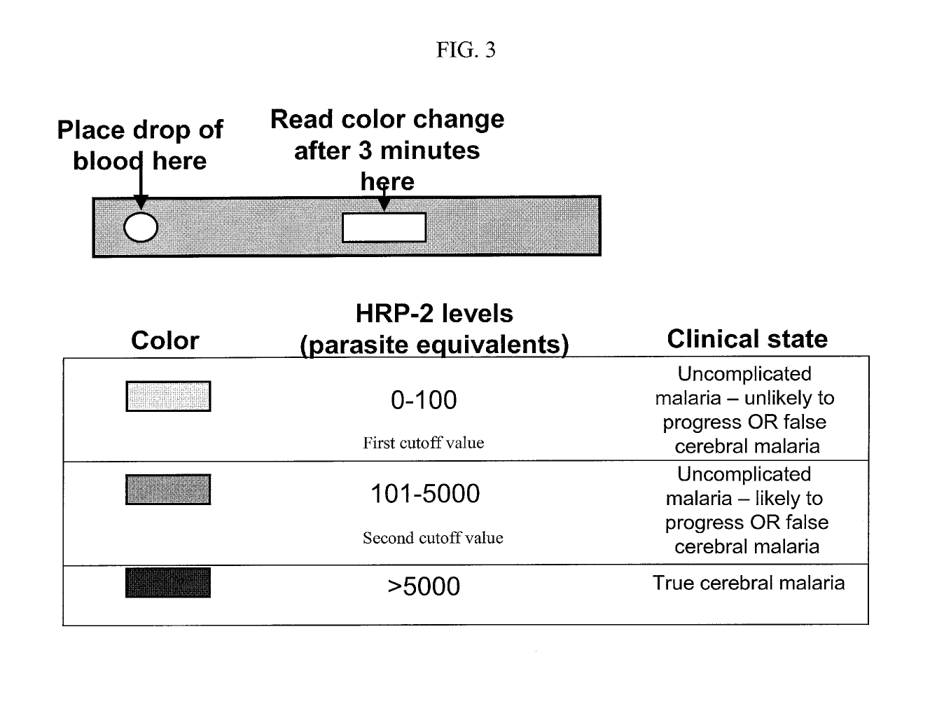

An exemplary test strip (such as a dipstick) is shown in FIG. 3. In one embodiment, the same diagnostic device is contemplated for use to identify (distinguish): 1) those patients likely to progress to severe malaria disease, and 2) true malaria from false malaria. The inventors contemplate a device of the present inventions comprising a diagnostic platform, comprising a dipstick, see, exemplary dipstick in FIG. 3. In one embodiment, the platform is capable of showing an amount of histidine rich protein-2 above a cutoff value of the present inventions for identifying a patient with cerebral malaria (i.e. true cerebral malaria) from patients comprising disease states selected from the group consisting of patients at risk of developing cerebral malaria (i.e. likely to progress to cerebral malaria), false cerebral malaria, uncomplicated malaria, unlikely to progress to cerebral malaria (i.e. unlikely to be at risk of developing cerebral malaria), unlikely to be at risk of developing cerebral malaria, and uninfected. For example, the amount of HRP2 detected using a platform of the present inventions representing a patient with true malaria produces a dark color, such as red, black, dark blue, and the like, as apposed to lighter colors, such as orange, orange-yellow, light grey, white, and the like, as represented in FIG. 3. In another embodiment, the platform provides at least two cutoff points, a first cutoff point and a second cutoff point, wherein said amount of histidine rich protein-2 above the first cutoff point identifies patients with true cerebral malaria. In another embodiment, the amount of histidine rich protein-2 is between the first cutoff point and the second cutoff point, such that the amount is below the first cutoff point and above the second cutoff point, for identifying a patient with malaria disease severity selected from the group consisting of a patient at risk of developing (progressing) to cerebral malaria (i.e. uncomplicated malaria likely to progress) and false cerebral malaria (i.e. a patient in a coma that does not have cerebral malaria but does have another cause of the comatose state). The amounts of HRP2 in this embodiment is shown by lighter colors, such as orange, orange-yellow, light grey, white, and the like, as represented in FIG. 3. In a further embodiment, when the amount of histidine rich protein-2 is below both the first cutoff point and the second cutoff point, the dipstick shows exemplary colors yellow, light grey, white, and the like for identifying patients with malaria selected from the group consisting of uncomplicated malaria not at risk for developing more severe disease, such as cerebral malaria, and false cerebral malaria.

The inventors contemplate methods of using a test strip of the present inventions in all areas of the world, in particular sub-Saharan Africa, where testing for malaria disease severity is necessary. Specifically, the inventors contemplate using a device for finger pricking a patient and collecting at least 10 ul of blood to contact the test strip, as shown in FIG. 3. Using methods, such as diffusion methods and reagents described in cited publications herein, the dipstick is capable of a color change relating to the amount of HRP2 present in the blood sample. Further contemplated is a color change of the test strip that preferably occurs in a few seconds to minutes, for example, after 10 seconds, after 20 seconds, after 30 seconds, even further after 1 minute, 2 minutes, 3 minutes, and greater than 3 minutes, up to 30 minutes, and up to 1 hour.

F. Kits For Use With Compositions And Methods of The Present Inventions.

The inventors contemplate kits comprising a diagnostic platform of the present inventions. Such kits and components would have a shelf life capable of providing an accurate diagnostic reading despite any one or more of extreme heat, extreme cold, and lengthy storage time. In preferred embodiments, kit components would remain viable after extended storage times, such as months to years, while stored unrefrigerated in sub-Saharan Africa. In some embodiments, said kits comprise a sterile device for obtaining a blood sample, such as a lancet, finger pricker, pen needle, etc. In some embodiments, said kits comprise instructions for using a diagnostic platform of the present inventions. In some embodiments, said kits comprise a diagnostic chart for interpreting results (read out) of the diagnostic platform. In a preferred embodiment, a diagnostic chart for interpreting results is capable of showing the viewer values below and above cutoff points for assistance in providing a diagnosis.

An example of a diagnostic platform for use in a kit is shown in FIG. 3 as described below. The inventors contemplate using the cut-off values generated using methods of the present inventions for establishing cutoff values for use in diagnostic devices, such as demonstrated in FIG. 3. For example, an exemplary first cutoff value is contemplated to separate values between 0-100 parasite equivalents (i.e. a yellow/light color) and 101-5000 parasite equivalents (i.e. an orange/medium color (in between light and dark colors). As another example, a second cutoff value is contemplated to separate values between 101-parasite equivalents and great than 5000 parasite equivalents (i.e. a red/dark color). Diagnostically this would translate into a clinical state wherein HRP2 values below the first cutoff value would indicate uncomplicated malaria, unlikely to progress to a more life threatening malaria disease or false cerebral malaria. Further, an HRP2 value above the first cutoff value would indicate a potential to progress to true cerebral malaria if not already a true cerebral malaria stage of disease. Even further, an HRP2 value above the first cutoff value and below the second cutoff value would indicate the patient has uncomplicated malaria, and malaria likely to progress to cerebral malaria, or false cerebral malaria (also unlikely to have retinal degeneration). Finally, an HRP2 value above the second cutoff value would indicate a true cerebral malaria stage of disease in a patient (also likely to have degeneration). Thus a device of the present inventions may have an embodiment with one cutoff value, an embodiment with two cutoff values, or cutoff value determined to be of diagnostic use in the present inventions.

### EXPERIMENTAL

The following examples serve to illustrate certain embodiments and aspects of the present invention and are not to be construed as limiting the scope thereof.

In the experimental disclosure which follows, the following abbreviations apply: N (normal); M (molar); mM (millimolar); μM (micromolar); mol (moles); mmol (millimoles); μmol (micromoles); nmol (nanomoles); pmol (picomoles); g (grams); mg (milligrams); μg (micrograms); ng (nanograms); 1 or L (liters); ml (milliliters); μl (microliters); cm (centimeters); mm (millimeters); μm (micrometers); nm (nanometers); ° C. (degrees Centigrade); MW (molecular weight in kd); k (kilo); dal and d (daltons).

### Example I

This Example describes exemplary Materials and Methods used during the discovery of a method for obtaining a cerebral malaria ROC cut-off point based upon an antibody test for detecting HRP2 proteins in the blood of malaria patients. This cut-off point in contemplated for use in embodiments of the present inventions for identifying cerebral malaria patients as a subset of patients with malaria parasitemia.

A Receiver Operating Characteristic curve (or ROC curve) was generated from children meeting the clinical case definition of cerebral malaria for use in diagnosing children with cerebral malaria.

Blood samples were obtained from children (patients) enrolled in a large autopsy based research study of cerebral malaria centered at Queen Elizabeth Central Hospital in Blantyre, Malawi (see, Taylor et al., Nature Medicine, 10:143-145 (2004), herein incorporated by reference, for further details). Children of the ages 6 months to 9 years that presented at the hospital were evaluated prior to death. Those children meeting the clinical case definition of cerebral malaria were enrolled in the study after consent was obtained from parent or guardian. This definition includes a Blantyre Coma Score of ≦2, and peripheral parasitemia with P. falciparum of any density. Exclusion criteria include other discernable causes of coma (i.e. hypoglycemia, meningitis, or post-ictal state). Blood samples were taken and frozen as lithium heparin anti-coagulated serum for long-term storage.

A test analysis of plasma HRP-2 (pHRP-2) levels was made by the inventors using 115 blood samples from patients evaluated by retinopathy (true malaria) compared to pHRP-2 levels in 146 samples from malaria patients that were negative for retinopathy (i.e. false malaria). Forty-eight samples from children with UCM were also included in the analysis. Exemplary dot plots of pHRP-2 levels and a ROC curve were generated from pHRP-2 levels as determined by ELISA as described below.

Frozen stocks of lithium heparin anti-coagulated serum were thawed. Serum was diluted at a ratio of either 1:200 or 1:500 in PBS. These samples, as well as a titration of a stock of known parasite concentration in duplicate, were plated onto a plate precoated with anti-HRP2 antibody (Cellabs, Brookvale, Australia, herein incorporated by reference). One hundred ul of serum sample were plated per well. The manufacturer's protocol (see, Kifude, et al., Clinical And Vaccine Immunology, June 2008, p. 1012-1018, herein incorporated by reference) was followed with the inventors' exception of incubation at 37oC in a humidified chamber. Briefly, a 1 hour sample incubation step was followed with extensive washing with PBS/0.1% Tween. 100 ul of the second conjugated antibody was plated and allowed to incubate for 1 hour. The conjugate was subsequently washed off and 100 ul of substrate was added for 15 minutes, during which color change was observed. This reaction was stopped with 50 ul of stop solution and the plate was analyzed at OD450. A standard curve relating OD to number of parasites was generated, and OD readings of the unknown samples were used to calculate parasite equivalents in original serum samples. An additional patient population was generated using patients admitted with uncomplicated malaria (UCM). This population contained children that had no alteration of consciousness, but were parasitemic at the time of admission. UCM serum samples were obtained from a microhematocrit tube in which 100 ul of blood was obtained at the time of admission. These tubes were spun in a microfuge at 1000 rpm for 5 minutes, after which the tubes were broken and serum removed. Serum was diluted in PBS as with the archived samples and the remainder of the analysis was identical.

Prior to obtaining the following test results, there was an expectation of an overlap between levels of pHRP2 across the malaria patient subgroups tested herein that would not be useful for distinguishing between subgroups of malaria patient. In part, these expectations were based upon known factors, such as the knowledge that in general malaria parasites produce HRP2 in the blood of parasitized patients combined with few known correlations between parasite load and particular subgroups of malaria patients.

Surprisingly, the inventors discovered during the development of the present inventions that HRP2 levels in blood plasma showed strong statistical correlations with both parasite load and a subgroup of child severe malaria patients with cerebral malaria.

Specifically, when patients were separated into groups, the CM retinopathy+ group showed significantly higher levels of HRP2 than either the CM retinopathy− or UCM patients. An exemplary data set obtained from the ELISA tests are shown in Table 1.

TABLE 1
pHRP-2 levels in serum from children with parasitemia as determined
by ELISA.

| | CM retinopathy + | CM Retinopathy − | UCM |
|---|---|---|---|
| parasite | Positive patients | Negative patients | Patients |
| equivalents/ul | pe/ul | pe/ul | pe/ul |
| Range | 700-37007 | 0-21857 | 0-5085 |
| Average | 13704 | 3132 | 517 |
| Median | 13147 | 1566 | 174 |
| | 700 | 0 | 0 |
| | 1437 | 10 | 0 |
| | 1775 | 17 | 0 |
| | 1821 | 30 | 0 |
| | 2058 | 30 | 0 |
| | 2544 | 30 | 0 |
| | 2578 | 30 | 0 |
| | 2759 | 40 | 6 |
| | 2838 | 50 | 6 |
| | 2951 | 60 | 15 |
| | 3037 | 68 | 16 |
| | 3102 | 70 | 18 |
| | 3355 | 101 | 20 |
| | 3699 | 109 | 21 |
| | 4187 | 147 | 31 |
| | 4254 | 163 | 38 |
| | 4265 | 195 | 50 |
| | 4298 | 240 | 70 |
| | 4580 | 250 | 71 |
| | 4648 | 263 | 119 |
| | 4896 | 263 | 127 |
| | 4905 | 265 | 149 |
| | 5010 | 278 | 156 |
| | 6232 | 293 | 171 |
| | 6345 | 297 | 177 |
| | 6531 | 299 | 184 |
| | 6655 | 304 | 211 |
| | 7069 | 316 | 217 |
| | 7544 | 347 | 223 |
| | 7555 | 349 | 224 |
| | 7583 | 361 | 245 |
| | 8008 | 386 | 250 |
| | 8291 | 424 | 253 |
| | 8834 | 440 | 311 |
| | 8979 | 489 | 314 |
| | 8996 | 532 | 341 |
| | 9015 | 566 | 412 |
| | 9225 | 569 | 431 |
| | 9309 | 594 | 452 |
| | 9592 | 603 | 454 |
| | 9727 | 606 | 711 |
| | 9999 | 644 | 1067 |
| | 10361 | 659 | 1094 |
| | 10666 | 725 | 1554 |
| | 10712 | 740 | 1835 |
| | 10842 | 747 | 2895 |
| | 10870 | 747 | 4795 |
| | 10870 | 763 | 5085 |
| | 11560 | 764 | |
| | 11809 | 770 | |
| | 11844 | 819 | |
| | 11857 | 960 | |
| | 11869 | 970 | |
| | 11919 | 1017 | |
| | 12137 | 1031 | |
| | 12345 | 1080 | |
| | 13110 | 1101 | |
| | 13147 | 1101 | |
| | 13147 | 1118 | |
| | 13257 | 1132 | |
| | 13279 | 1133 | |
| | 13323 | 1214 | |
| | 13561 | 1224 | |
| | 13630 | 1247 | |
| | 13887 | 1278 | |
| | 14275 | 1288 | |
| | 14422 | 1319 | |
| | 14535 | 1332 | |
| | 14626 | 1417 | |
| | 15644 | 1463 | |
| | 15938 | 1470 | |
| | 16153 | 1540 | |
| | 16583 | 1566 | |
| | 16857 | 1583 | |
| | 16922 | 1617 | |
| | 17020 | 1629 | |
| | 17057 | 1670 | |
| | 17132 | 1688 | |
| | 17227 | 1709 | |
| | 17352 | 1740 | |
| | 17400 | 1786 | |
| | 17533 | 1898 | |
| | 17816 | 1914 | |
| | 17984 | 2055 | |
| | 18381 | 2097 | |
| | 18393 | 2132 | |
| | 18511 | 2217 | |
| | 19112 | 2220 | |
| | 19137 | 2370 | |
| | 19288 | 2491 | |
| | 19350 | 2532 | |
| | 19592 | 2555 | |
| | 19802 | 2594 | |
| | 20259 | 2657 | |
| | 20304 | 2678 | |
| | 20654 | 2739 | |
| | 21175 | 2826 | |
| | 21380 | 2863 | |
| | 22634 | 2947 | |
| | 22738 | 3042 | |
| | 22746 | 3140 | |
| | 22804 | 3183 | |
| | 22861 | 3429 | |
| | 22974 | 3438 | |
| | 23761 | 3482 | |
| | 23764 | 3555 | |
| | 24463 | 3678 | |
| | 26673 | 3727 | |
| | 26789 | 3886 | |
| | 26910 | 4168 | |
| | 28310 | 4278 | |
| | 28556 | 4286 | |
| | 34223 | 4324 | |
| | 36606 | 4513 | |
| | 36894 | 4632 | |
| | 37007 | 4840 | |
| | | 4924 | |
| | | 5189 | |
| | | 5542 | |
| | | 5686 | |
| | | 5882 | |
| | | 6017 | |
| | | 6047 | |
| | | 7117 | |
| | | 7294 | |
| | | 7578 | |
| | | 8052 | |
| | | 8078 | |
| | | 8824 | |
| | | 9060 | |
| | | 9063 | |
| | | 9078 | |
| | | 10224 | |
| | | 10733 | |
| | | 11332 | |
| | | 12463 | |
| | | 13427 | |
| | | 13532 | |
| | | 14535 | |
| | | 15169 | |
| | | 16343 | |
| | | 16518 | |
| | | 16526 | |
| | | 19140 | |
| | | 21857 | |

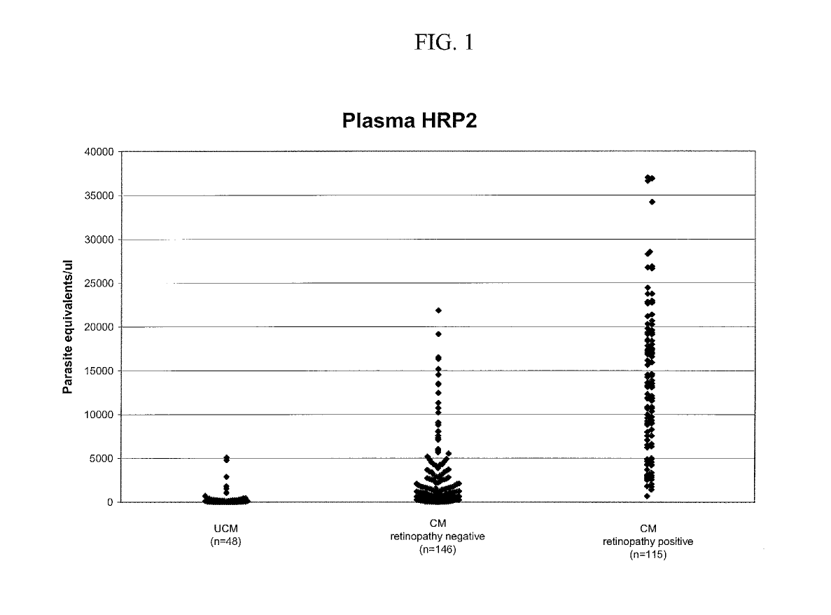

For analysis, pHRP-2 levels in serum from retinopathy positive (true malaria) patients, using 115 children's blood samples, were compared to HRP-2 levels from retinopathy negative (false malaria) patients' blood serum, 146 children's blood samples. Blood samples from 48 children with UCM were also included in the analysis. Exemplary dot plots of pHRP-2 levels between these two groups were generated and shown in FIG. 1.

The values obtained in Table 1 were then statistically evaluated by SPSS (Statistical Package for the Social Sciences) for generating ROC curves and determining relevant areas under the curve (AUC).

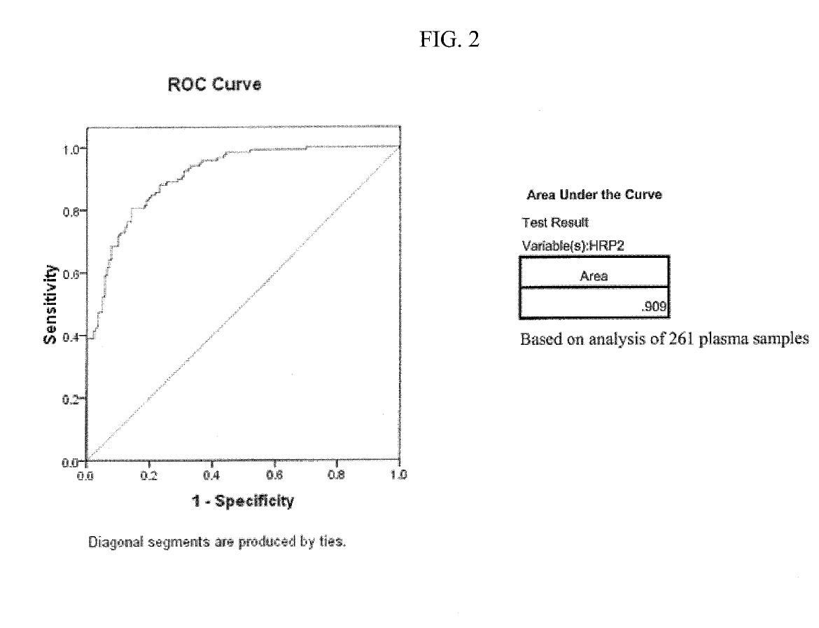

SPSS analysis demonstrated the capability of determining a highly specific and highly accurate cut-off value using HRP2 values that correlated with patients previously diagnosed with CM retinopathy, see, Table 2 and FIG. 2.

Further, this data demonstrated that patient subgroups with UCM were capable of being separated from CM and NCM patient groups using the ROC curve cut point values provided with methods of the present inventions.

TABLE 2
Optimal calculation of Area Under the ROC curve cut off values at a CI
of 95%, see, numbers in BOLD. The curve values generated are shown in
graphical form in FIG. 2.
Coordinates of the Curve
Test Result Variable(s): HRP2all

| Positive if Greater Than or | Sensitivity | 1 - Specificity |
|---|---|---|
| Equal To^{a} | True positive | False positive |
| (Cutpoint) | (115 total) | (143 total) |
| 9.00 | 1.000 | 1.000 |
| 13.50 | 1.000 | .993 |
| 18.50 | 1.000 | .986 |
| 25.00 | 1.000 | .979 |
| 35.00 | 1.000 | .951 |
| 45.00 | 1.000 | .944 |
| 55.00 | 1.000 | .937 |
| 64.00 | 1.000 | .930 |
| 69.00 | 1.000 | .923 |
| 85.50 | 1.000 | .916 |
| 105.00 | 1.000 | .909 |
| 128.00 | 1.000 | .902 |
| 155.00 | 1.000 | .895 |
| 179.00 | 1.000 | .888 |
| 217.50 | 1.000 | .881 |
| 245.00 | 1.000 | .874 |
| 256.50 | 1.000 | .867 |
| 264.00 | 1.000 | .853 |
| 271.50 | 1.000 | .846 |
| 285.50 | 1.000 | .839 |
| 295.00 | 1.000 | .832 |
| 298.00 | 1.000 | .825 |
| 301.50 | 1.000 | .818 |
| 310.00 | 1.000 | .811 |
| 331.50 | 1.000 | .804 |
| 348.00 | 1.000 | .797 |
| 355.00 | 1.000 | .790 |
| 373.50 | 1.000 | .783 |
| 405.00 | 1.000 | .776 |
| 432.00 | 1.000 | .769 |
| 464.50 | 1.000 | .762 |
| 510.50 | 1.000 | .755 |
| 549.00 | 1.000 | .748 |
| 567.50 | 1.000 | .741 |
| 581.50 | 1.000 | .734 |
| 598.50 | 1.000 | .727 |
| 604.50 | 1.000 | .720 |
| 625.00 | 1.000 | .713 |
| 651.50 | 1.000 | .706 |
| 679.50 | 1.000 | .699 |
| 712.50 | .992 | .699 |
| 732.50 | .992 | .692 |

The test result variable(s): HRP2all has at least one tie between the positive actual state group and the negative actual state group.
^{a}The smallest cutoff value is the minimum observed test value minus 1, and the largest cutoff value is the maximum observed test value plus 1. All the other cutoff values are the averages of two consecutive ordered observed test values.

In order to validate the ROC curve, a smaller group of patients were grouped as CM retinopathy positive vs. CM negative patients, see Table 3A, and evaluated by generating an ROC curve with an optimal AUC. Exemplary values for an AUC and a ROC are shown in Tables 3B and C, respectively, below.

TABLE 3A
ROC curve points generated by SPSS using pHRP-2 levels measured
by ELISA comparing patients with “true malaria” (retinopathy positive) to
patients with “false malaria” retinopathy negative.

| CM retinopathy + | CM |
|---|---|
| Positive^{a} patients | Negative patients |
| 76 | 99 |

Larger values of the test result variable(s) indicate stronger evidence for a positive actual state.
^{a}The positive actual state is 1.00.

TABLE 3B
Optimal calculation of Area Under the ROC curve at a confidence
interval (CI) of 95% and corresponding cut-off point determination.
Area Under the Curve
Test Result Variable(s): HRP2
Area
0.929

TABLE 3C
Exemplary coordinate results for calculating the Area Under the ROC
curve at a CI of 95% and corresponding cut-off point determination, see,
numbers in BOLD.
Coordinates of the Curve
Test Result Variable(s): HRP2

| | Sensitivity | 1 - Specificity |
|---|---|---|
| Positive if Greater Than | CM retinopathy + | CM Retinopathy − |
| or Equal To^{a} | Positive^{a} patients | Negative patients |
| 9.0000 | 1.000 | 1.000 |
| 13.5000 | 1.000 | .990 |
| 18.5000 | 1.000 | .980 |
| 25.0000 | 1.000 | .970 |
| 35.0000 | 1.000 | .929 |
| 45.0000 | 1.000 | .919 |
| 59.0000 | 1.000 | .909 |
| 69.0000 | 1.000 | .899 |
| 89.5000 | 1.000 | .889 |
| 128.0000 | 1.000 | .879 |
| 155.0000 | 1.000 | .869 |
| 179.0000 | 1.000 | .859 |
| 217.5000 | 1.000 | .848 |
| 245.0000 | 1.000 | .838 |
| 256.5000 | 1.000 | .828 |
| 270.5000 | 1.000 | .808 |
| 285.5000 | 1.000 | .798 |
| 295.0000 | 1.000 | .788 |
| 298.0000 | 1.000 | .778 |
| 301.5000 | 1.000 | .768 |
| 310.0000 | 1.000 | .758 |
| 331.5000 | 1.000 | .747 |
| 348.0000 | 1.000 | .737 |
| 367.5000 | 1.000 | .727 |
| 413.0000 | 1.000 | .717 |
| 486.0000 | 1.000 | .707 |
| 549.0000 | 1.000 | .697 |
| 580.0000 | 1.000 | .687 |
| 598.5000 | 1.000 | .677 |
| 604.5000 | 1.000 | .667 |
| 632.5000 | 1.000 | .657 |
| 679.5000 | 1.000 | .646 |
| 720.0000 | .987 | .646 |

^{a}The smallest cutoff value is the minimum observed test value minus 1, and the largest cutoff value is the maximum observed test value plus 1. All the other cutoff values are the averages of two consecutive ordered observed test values.

Therefore, the inventors showed that plasma HRP2 measurements, using antibodies for binding to HRP2, were capable of being used to provide ROC curve cut-off values useful for identifying subgroups of comatose patients that had cerebral malaria, as opposed to other patients with incidental parasitemia that were in comas due to non-malarial causes.

The inventors contemplate, in one embodiment, using the cut-off values generated using methods of the present inventions for establishing cutoff values for use in diagnostic devices, such as demonstrated in FIG. 3. For example, an exemplary first cutoff value is contemplated to separate values between 0-100 parasite equivalents (i.e. a yellow/light color) and 101-5000 parasite equivalents (i.e. an orange/medium color (in between light and dark colors). As another example, a second cutoff value is contemplated to separate values between 101-parasite equivalents and great than 5000 parasite equivalents (i.e. a red/dark color). Diagnostically this would translate into a clinical state wherein HRP2 values below the first cutoff value would indicate uncomplicated malaria, unlikely to progress to a more life threatening malaria disease or false cerebral malaria. Further, an HRP2 value above the first cutoff value would indicate a potential to progress to true cerebral malaria if not already a true cerebral malaria stage of disease. Even further, an HRP2 value above the first cutoff value and below the second cutoff value would indicate the patient has uncomplicated malaria, and malaria likely to progress to cerebral malaria, or false cerebral malaria (also unlikely to have retinal degeneration). Finally, an HRP2 value above the second cutoff value would indicate a true cerebral malaria stage of disease in a patient (also likely to have degeneration). Thus a device of the present inventions may have an embodiment with one cutoff value, an embodiment with two cutoff values, or cutoff value determined to be of diagnostic use in the present inventions.

### Example II

This Example describes a contemplated exemplary diagnostic device for use with said Receiver Operating Characteristic curves (ROC) of the present inventions.

A hand-held diagnostic immunoassay device capable of providing a plasma HRP2 level for use in categorizing patients, in part for use in supporting or providing a clinical diagnosis, is contemplated for use. In a preferred embodiment, said device is for use in regions without diagnostic clinical capabilities, such as in sub-Saharan Africa, and for use during travel, for use when suspecting parasitism, and the like. Specifically, a handheld diagnostic device with a quick immunoassay read out (on the order of minutes to a few hours) for determining plasma HRP2 levels is contemplated. Such a device comprises a test strip, wherein an antibody for specifically binding to HRP2 is attached to said test strip, and a colorimetric read-out for use in combination with a read-out chart, comprising cut-off values for identifying subcategories of patients, wherein said cut-offs are determined by an ROC curve of the present inventions. An exemplary test strip is shown in FIG. 3. In one embodiment, the same diagnostic device can be used to distinguish: 1) those patients likely to progress to severe disease, and 2) true from false malaria.

An exemplary handheld device is a dipstick, such as analogous to a dipstick for use in determining pregnancy in a human. Examples of such diagnostic devices are described in United States Patent Application No. 20060281188, U.S. Pat. No. 7,192,701, and devices comprising antibodies, including antibody generation for use in such devices, for example, as described in U.S. Pat. No. 6,716,641, United States Patent Application Nos. 20090305436, 20090258375, 20070248962, 20040146900 and kits in U.S. Pat. Nos. 7,390,489, 6,905,885, 6,716,641, and the like, all of which are herein incorporated by reference.

Further, a device of the present inventions would further comprise a positive control for establishing that the device is capable of providing a read-out (i.e. shelf life has not expired). For example, a device may have an antibody for reacting to a serum albumen protein found in the blood of every human. Even further, the inventors contemplate a kit comprising a device (diagnostic platform) of the present inventions. Yet even further, the kit comprises instructions for using said diagnostic platform.

### Example III

This Example describes the determination of the relationship of indictors in studies of retinopathy-positive and retinopathy-negative cerebral malaria. The studies included: 1. Cases with autopsy confirmation of disease state; 2. A retrospective study using retinopathy as surrogate marker for disease state; and 3. A prospective study including all patients admitted with clinically defined cerebral malaria during a single season.

In this Example ELISA was performed on diluted plasma samples with recombinant protein used as standard to generate quantification. Nanograms of Histidine Rich Protein—2 per mL plasma (ng HRP2/ml plasma) was used as standard to generate quantification. Previously the standard was based up on parasite equivalents/μL plasma.

1. Cases with Autopsy Confirmation of Disease State

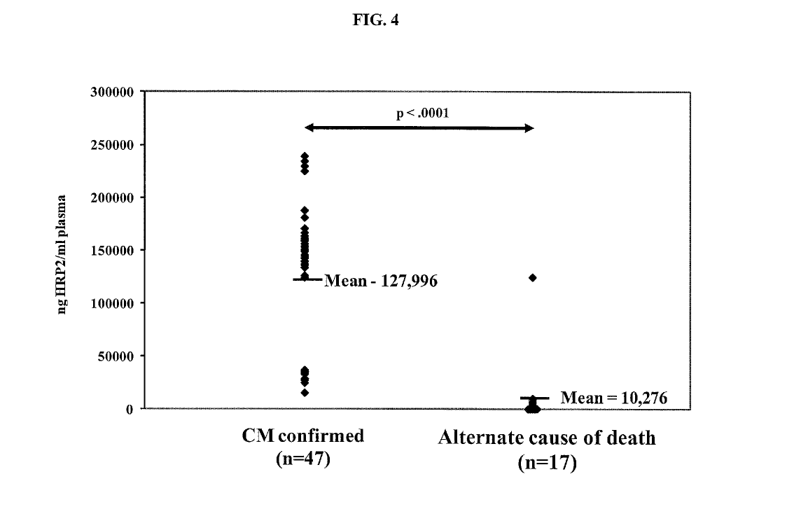

Archived plasma samples from patients who met the clinical case definition of cerebral malaria, died, and went on to autopsy (1996-2008) were evaluated for ng HRP2/ml plasma. Of the 64 total cases, 47 were found with pathologic confirmation of cerebral malaria and 17 were found with no evidence of cerebral malaria (and another cause of death). FIG. 4 illustrates the distribution of ng HRP2/ml plasma for the autopsy cases found with pathologic confirmation of cerebral malaria and with no evidence of cerebral malaria. The mean for pathologic confirmation of cerebral malaria was 127,996 ng HRP2/ml plasma. The mean for cases found with no evidence of cerebral malaria was 10,276 ng HRP2/ml plasma.

2. Retrospective Study Using Retinopathy as Surrogate Marker for Disease State

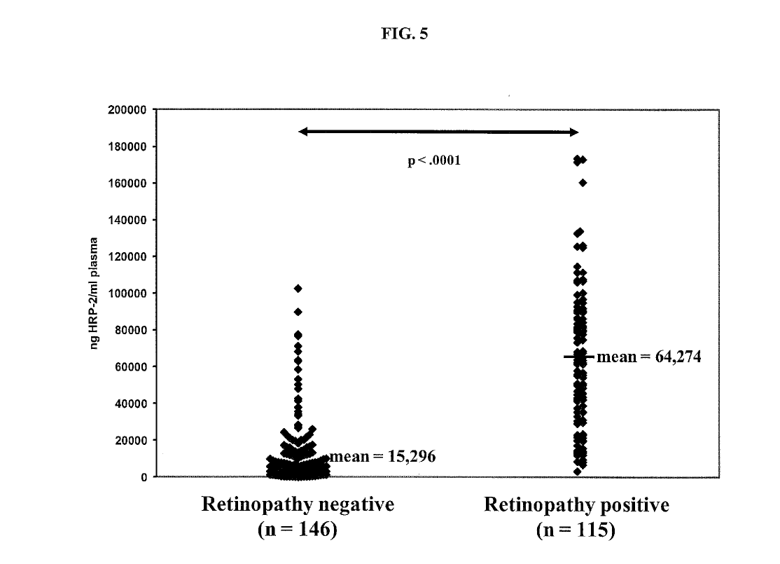

Archived plasma samples from patients who met the clinical case definition for cerebral malaria and had ophthalmological examinations (2000-2008) were analyzed for ng HRP2/ml plasma. Malaria retinopathy has long been the standard by which the diagnosis of cerebral malaria is confirmed. Of the 261 total samples, 115 of the patients were found with malaria retinopathy and 146 of the patients were found without malaria retinopathy. FIG. 5 illustrates the distribution of ng HRP2/ml plasma for patients found with and without malaria retinopathy. The mean for patients diagnosed with malaria retinopathy was 64,274 ng HRP2/ml plasma. The mean for patients diagnosed without malaria retinopathy was 15,296 ng HRP2/ml plasma.

3. Prospective Study Including all Patients Admitted with Clinically Defined Cerebral Malaria During a Single Season

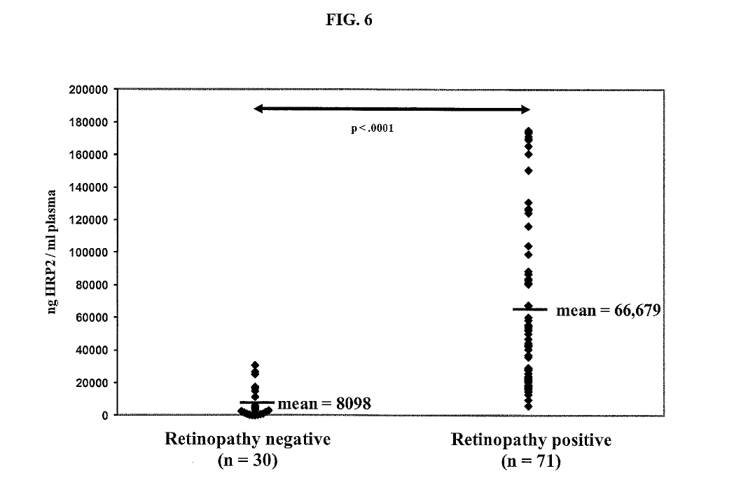

Plasma samples from patients who met the clinical case definition for cerebral malaria, had ophthalmological examinations, and were admitted during the 2009 season were analyzed for ng HRP2/ml plasma. Of the 101 patients, 71 were found with retinopathy and 30 without retinopathy. FIG. 6 illustrates the distribution of ng HRP2/ml plasma for patients found with and without malaria retinopathy. The mean for patients diagnosed with malaria retinopathy was 66,679 ng HRP2/ml plasma. The mean for patients diagnosed without malaria retinopathy was 8,098 ng HRP2/ml plasma.

### Determination of a Possible Cut-Offs for CM Diagnostic Application:

| | |
|---|---|
| Retinopathy | Retinopathy |
| positive | negative |

10,000 ng/ml cut-off

| | | | |
|---|---|---|---|
| HRP-2 | 70 | 11 | Sensitivity: 97 |
| positive | | | Specificity: 66 |
| HRP-2 | 2 | 22 | PPV: 86 |
| negative | | | NPV: 91 |

14,000 ng/ml

| | | | |
|---|---|---|---|
| HRP-2 | 69 | 8 | Sensitivity: 95 |
| positive | | | Specificity: 75 |
| HRP-2 | 3 | 25 | PPV: 89 |
| negative | | | NPV: 89 |

20,000 ng/ml

| | | | |
|---|---|---|---|
| HRP-2 | 60 | 4 | Sensitivity: 83 |
| positive | | | Specificity: 87 |
| HRP-2 | 4 | 29 | PPV: 93 |
| negative | | | NPV: 70 |

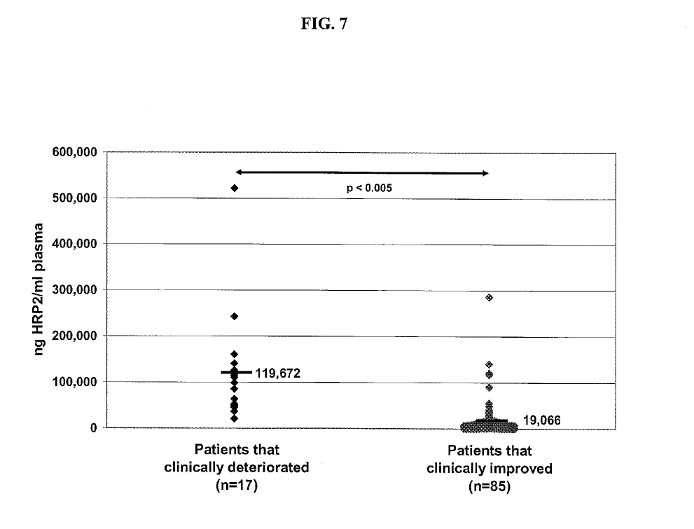

The HRP2/ml plasma concentration was subsequently analyzed for 102 patients and analyzed within the context of the patient outcome. FIG. 7. illustrates the distribution of HRP2/ml plasma concentration for the 85 patients who improved and the 17 patients who clinically deteriorated. The mean ng HRP2/ml plasma for patients who improved was 19,066. The mean ng HRP2/ml plasma for patients who clinically deteriorated was 119,672.

### Possible Cutoffs for Prognostic Application

| | |
|---|---|
| Deteriorate | Improve |

20,000 ng/ml

| | | | |
|---|---|---|---|
| HRP-2 | 17 | 20 | Sensitivity: 100 |
| positive | | | Specificity: 76 |
| HRP-2 | 0 | 65 | |
| negative | | | |

30,000 ng/ml

| | | | |
|---|---|---|---|
| HRP-2 | 16 | 13 | Sensitivity: 94 |
| positive | | | Specificity: 84 |
| HRP-2 | 1 | 72 | |
| negative | | | |

40,000 ng/ml

| | | | |
|---|---|---|---|
| HRP-2 | 15 | 8 | Sensitivity: 88 |
| positive | | | Specificity: 90 |
| HRP-2 | 2 | 77 | |
| negative | | | |

These examples support the use of HRP2/ml plasma as a diagnostic method of determining true cerebral malaria and use of HRP2/ml plasma as a prognostic method for determining those patients that are likely to deteriorate clinically.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in human medicine, immunology, chemistry, molecular biology, biochemistry, or related fields are intended to be within the scope of the following claims.

1. A method, comprising:
a) providing,
i) blood from a human, and
ii) an antibody reactive with histidine rich protein-2,
b) reacting said antibody with said blood under conditions such that the amount of histidine rich protein-2 in the blood is quantitated; and
c) determining whether the quantitated amount of histidine rich protein-2 (HRP-2) is below or above a cutoff value.

2. The method of claim 1, wherein said human is a comatose child.

3. The method of claim 2, wherein prior to reacting of step b), said child is tested for meningitis.

4. The method of claim 1, further comprising the step of diagnosing cerebral malaria in said human when the quantitated amount is above said cutoff value.

5. The method of claim 1, further comprising the step of treating cerebral malaria in said human when the quantitated amount is above said cutoff value.

6. The method of claim 5, wherein said treating comprises administering antimalaria chemotherapy.

7. The method of claim 1, wherein said antibody is attached to a solid support.

8. The method of claim 7, wherein said solid support is a diagnostic platform, or dipstick.

9. The method of claim 8, wherein said diagnostic platform, miniaturized lateral flow device, or dipstick comprises recombinant HRP-2 antibodies.

10. The method of claim 9, where in the amount of HRP-2 in the blood is quantitated in terms of ng/ml HRP-2.

11. The method of claim 1, wherein the cutoff value separates values between 0-100 parasite equivalents per microliter and 101-5000 parasite equivalents per microliter.

12. The method of claim 1, wherein the cutoff value separates values between 101-5000 parasite equivalents per microliter and greater than 5000 parasite equivalents per microliter.

13. The method of claim 1, further comprising the step of diagnosing the absence of cerebral malaria in said human when the quantitated amount is below the cutoff value.

14. The method of claim 1, further comprising the step of treating said human for a disease other than cerebral malaria when the quantitated amount is below said cutoff value.

15. The method of claim 8, wherein the diagnostic platform or dipstick reads negative when said quantitated amount of histidine rich protein-2 is below said cutoff value.

16. The method of claim 8, wherein the diagnostic platform or dipstick reads positive when said quantitated amount of histidine rich protein-2 is above said second cutoff value.

17. The method of claim 8, wherein said diagnostic platform or dipstick comprises a miniaturized lateral flow device, or an immuno-chromatographic test strip.

18. The method of claim 8, wherein said diagnostic platform or dipstick comprises a fluid-receiving zone.

19. A kit comprising a diagnostic platform, said diagnostic platform capable of quantitating histidine rich protein 2 in a blood sample, and instructions, said instructions providing a cut-off value, above which a quantified histidine rich protein-2 level in a blood sample indicates cerebral malaria.

20. The kit of claim 19, wherein the diagnostic platform reads negative when a quantified histidine rich protein-2 level is below the cutoff value.

21. The kit of claim 19, wherein the diagnostic platform reads positive when a quantified histidine rich protein-2 level is above the cutoff value.

22. The kit of claim 19, wherein the diagnostic platform comprises a membrane, a charged membrane, plastic, beads, strips, microtiter wells, microchannels or a combination thereof.

23. The kit of claim 19, wherein said diagnostic platform comprises an immuno-chromatographic test strip, miniaturized lateral flow device, or enzyme-linked immunosorbent assay platform.

24. The kit of claim 19, wherein said diagnostic platform comprises a fluid-receiving zone.

25. The kit of claim 19, wherein said diagnostic platform is adapted for quantifying histidine rich protein-2 in plasma or serum from the blood sample.

26. The kit of claim 19, wherein the cut-off values separates values between 0-100 parasite equivalents per microliter and 101-5000 parasite equivalents equivalents per microliter.

27. The kit of claim 19, wherein the cut-off value separates values between 101-5000 parasite equivalents per microliter and greater than 5000 parasite equivalents per microliter.

28. The method of claim 1, wherein determining whether the quantitated amount of histidine rich protein-2 is above a cutoff value identifies a blood sample as being from a human at risk for progression of relatively mild malaria disease to the more life-threatening malaria disease.

29. The method of claim 28, wherein the more life-threatening malaria disease is cerebral malaria.

30. The method of claim 28, wherein plasma or serum from the blood sample is reacted with the antibody.

31. A method, comprising,
a) providing a diagnostic platform that distinguishes an amount of histidine rich protein-2 above a cutoff value in blood from a patient having cerebral malaria from a control histidine rich protein-2 amount in blood from a patient with malaria who does not have cerebral malaria;
b) contacting said diagnostic platform with a blood sample under conditions for obtaining a quantitative value for an amount of histidine rich protein-2 in blood from a patient diagnosed with cerebral malaria retinopathy; and
c) determining whether said amount of histidine rich protein-2 in said blood sample is above or below the cutoff value to thereby identify a patient with a risk for progression of relatively mild malaria disease to the more life-threatening cerebral malaria having cerebral malaria from a patient that does not have cerebral malaria.

32. The method of claim 31, wherein the patient is comatose.

33. The method of claim 31, further identifying whether a comatose patient does not have cerebral malaria.

34. The method of claim 31, wherein the cut-off value was generated from a receiver operating characteristic curve that identifies patients having cerebral malaria retinopathy from patients that do not have cerebral malaria retinopathy.

35. The method of claim 31, wherein the cut-off value separates values between 0-100 parasite equivalents per microliter and 101-5000 parasite equivalents equivalents per microliter.

36. The method of claim 31, wherein the cut-off value separates values between 101-5000 parasite equivalents per microliter and greater than 5000 parasite equivalents per microliter.

37. The method of claim 31, wherein the diagnostic platform is contacted with plasma or serum from the blood sample.

38. The method of claim 31, further comprising treating cerebral malaria in the patient when the amount of histidine rich protein-2 in the patient's blood sample is above the cutoff value.

39. The method of claim 31, further comprising treating the patient when the amount of histidine rich protein-2 in the patient's blood sample is below the cutoff value and when the patient is comatose.
